(19) United States
(12) Patent Application Publication
(10) Pub. No.: US 2010/0093745 A1
(43) Pub. Date: Apr. 15, 2010

(54) THERAPEUTIC AGENT OR PROPHYLACTIC AGENT FOR DEMYELINATING DISEASE COMPRISING AMINO ALCOHOL DERIVATIVE AS ACTIVE INGREDIENT

(76) Inventors: Kazuhiko Kuriyama, Tochigi (JP); Tokutarou Yasue, Tochigi (JP)

Correspondence Address: WENDEROTH, LIND & PONACK, L.L.P., 1030 15th Street, N.W.,, Suite 400 East, Washington, DC, 20005-1503, US

(21) Appl. No.: 12/449,517

(22) PCT Filed: Feb. 8, 2008
(86) PCT No.: PCT/JP2008/052138

§ 371 Date: Aug. 12, 2009

(30) Foreign Application Priority Data
Feb. 13, 2007 (JP) 2007-031718

(51) Int. Cl. A61K 31/4965; A61K 31/095; A61K 31/235; A61P 25/28; A61P 21/00

(52) U.S. Cl. 514/252.1; 514/706; 514/532

(57) ABSTRACT

A novel therapeutic agent or prophylactic agent for a demyelinating disease is provided. An amino alcohol derivative represented by the general formula (1),

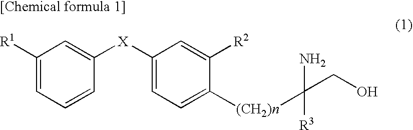

which is a sphingosine-1-phosphate receptor agonist, a pharmacologically acceptable salt or hydrates thereof, are a therapeutic agent or prophylactic agent for a demyelinating disease.

### TECHNICAL FIELD

The present invention relates to a therapeutic agent for a demyelinating disease, which comprises an amino alcohol derivative or a pharmacologically acceptable salt or hydrate thereof as an active ingredient, or a method of treating a demyelinating disease.

### BACKGROUND OF THE INVENTION

The demyelinating disease is a serious disease of brain and spinal cord, including damage of myelin sheath encircling a nerve fiber. As a result of demyelination or myelitis or optic neuritis, various nervous symptoms including movement disorder, visual loss and sensory impairment are generated. Multiple sclerosis (MS) is the most typical case of the demyelinating disease.

MS is characterized by various symptoms and signs of central nervous system insufficiency which accompanies remission and repeating recrudescence. Most frequent incipient symptoms are abnormal sensation of the upper and lower limbs, the body and unilateral face; paralysis and clumsy lower limbs and hands; and vision disorders such as partial blindness and pain of one eye (retrobulbar optic neuritis), blurred vision, scotoma and the like. Other frequently found incipient symptoms include eye muscle paralysis which induces double vision, transient muscular strength reduction of a limb or limbs, slight stiffness of limbs or abnormal fatigue, slight gait disturbance, difficulty of bladder control, vertigo and slight emotional disturbance. All of these are a proof that the central nervous system was diffusively violated and are frequently expressed from several months to several years before recognition of the disease.

Therapeutic drugs of MS are divided into treatment of acute exacerbation phase and recurrence inhibitor. The treatment of acute exacerbation phase has been almost established, and a so-called pulse therapy in which intravenous drip infusion of 1,000 mg methylprednisolone is carried out for 3 to 5 days is generally selected (Non-patent References 1 and 2). It has been revealed that a steroid pulse therapy significantly quickens recovery of dropped out nervous symptoms, but the effect of reducing aftereffect of the disease cannot be expected (Non-patent Reference 2). Thus, a plasma exchange therapy from which the effect for reducing the symptoms can be expected has been drawing attention in recent years (Non-patent Reference 3). On the other hand, it is known that the steroid pulse therapy which is periodically carried out once in several months has an effect to inhibit the progressive cerebral atrophy found in some MS patients, but there also is a fact that recurrence of MS cannot be prevented (Non-patent Reference 4). As a result of carrying out clinical trials for the purpose of developing recurrence inhibitors, the recurrence preventive effect has been found also in the clinical trials of interferon-β1b every other day subcutaneous injections and once a week intramuscular injections and three times a week subcutaneous injections of interferon-β1a (Non-patent References 5 to 8). In addition, it has been reported during the same period that a particular polypeptide pharmaceutical preparation, glatiramer ascetate, induces the same effect at a similar degree of interferon-β by daily subcutaneous injection (Non-patent Reference 9). However, since these drugs are injections, they force pain upon the MS patients who require long-term use for the purpose of obtaining recurrence protection and progress prevention. The immunosuppressants for the serious progressive cases (methotrexate, azathioprine, cyclophosphamide, cladiribine) are not always uniformly effective but have a danger of considerable toxicity. Accordingly, concern has been directed toward the development of an orally administrable highly safe agent which alleviates neurological disorders that will be accumulated extending to the future and improves long-term prognosis. As such an orally administrable agent, fingolimod has been reported (Non-patent Reference 10). However, it has not been known on the usefulness of the amino monoalcohol derivative described in the present application as a therapeutic agent for a demyelinating disease.

Non-patent Reference 1: New Engl. J. Med., 1997, 337: 1604-1611

### Non-patent Reference 2: Neurology, 2002, 58: 169-178

Non-patent Reference 3: Ann. Neurol., 1999, 46: 878-886

### Non-patent Reference 4: Neurology, 2001, 57: 1239-1247

### Non-patent Reference 5: Neurology, 1993, 43: 655-661

### Non-patent Reference 6: Neurology, 1993, 43: 662-667

Non-patent Reference 7: Ann. Neurol., 1996, 39: 285-294

### Non-patent Reference 8: Lancet, 1998, 352: 1498-1504

### Non-patent Reference 9: Neurology, 1995, 45: 1268-1276

Non-patent Reference 10: New Engl. J. Med., 2006, 355: 1124-1140

### DISCLOSURE OF THE INVENTION

### Problems that the Invention is to Solve

An object of the invention is to provide a therapeutic agent for a demyelinating disease, which comprises an amino alcohol derivative, a pharmacologically acceptable salt or hydrate thereof as an active ingredient, or a method of treating a demyelinating disease.

### Means for Solving the Problems

The inventors have accomplished the invention by finding that an amino alcohol derivative is useful for the treatment of demyelinating diseases (multiple sclerosis, acute disseminated encephalomyelitis, adreno-leukodystrophy and adrenomyeloneuropathy, Leber hereditary optic atrophy, or human T lymphotropic viral myelopathy).

Specifically, the present invention relates to:

1) A therapeutic agent or prophylactic agent for a demyelinating disease, comprising as an active ingredient an amino alcohol derivative, or a pharmaceutically acceptable salt or hydrate thereof, wherein the amino alcohol derivative is represented by the general formula (1),

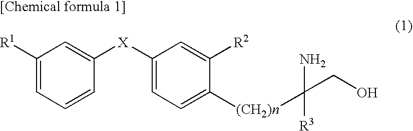

[wherein R¹ represents a chlorine atom or a straight-chain alkyl group having 1 to 3 carbon atoms or trifluoromethyl group, R² represents a fluorine atom or a chlorine atom, R³ represents a straight-chain alkyl group having 1 to 3 carbon atoms, X represents an oxygen atom or a sulfur atom, and n denotes 2 or 3].
2) The therapeutic agent or prophylactic agent for a demyelinating disease, comprising as an active ingredient the amino alcohol derivative according to 1), or a pharmaceutically acceptable salt or hydrate thereof, wherein the compound represented by the general formula (1) is a compound represented by the general formula (1a),

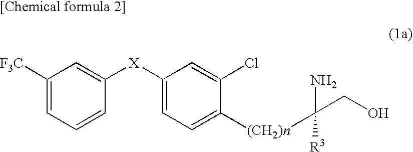

[wherein R³, X, and n are as described above].
3) The therapeutic agent or prophylactic agent for a demyelinating disease, comprising as an active ingredient the amino alcohol derivative according to 1) or 2), or a pharmaceutically acceptable salt or hydrate thereof, wherein in the general formulae (1) or (1a), R³ is a methyl group.
4) The therapeutic agent or prophylactic agent for a demyelinating disease, comprising as an active ingredient the amino alcohol derivative according to 1), or a pharmaceutically acceptable salt or hydrate thereof, wherein the compound represented by the general formula (1) is,
(R)-2-amino-5-[2-chloro-4-(3-trifluoromethylphenoxy)phenyl]-2-methylpentan-1-ol,
(R)-2-amino-5-[2-chloro-4-(3-trifluoromethylphenylthio)phenyl]-2-methylpentan-1-ol,
(R)-2-amino-4-[2-chloro-4-(3-trifluoromethylphenoxy)phenyl]-2-methylbutan-1-ol,
(R)-2-amino-4-[2-chloro-4-(3-trifluoromethylphenylthio)phenyl]-2-methylbutan-1-ol,
(R)-2-amino-5-[2-chloro-4-(3-ethylphenylthio)phenyl]-2-methylpentan-1-ol,
(R)-2-amino-5-[2-fluoro-4-(3-trifluoromethylphenylthio)phenyl]-2-methylpentan-1-ol, or
(R)-2-amino-5-[2-chloro-4-(3-trifluoromethylphenylthio)phenyl]-2-propylpentan-1-ol.
   5) A therapeutic agent or prophylactic agent for a demyelinating disease, comprising as an active ingredient an optically active amino alcohol derivative, or a pharmaceutically acceptable salt or hydrate thereof, which is obtainable by a step of allowing a compound represented by the general formula (2),

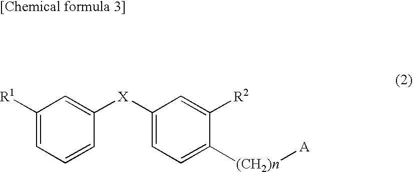

[wherein R¹ represents a chlorine atom or a straight-chain alkyl group having 1 to 3 carbon atoms or trifluoromethyl group, R² represents a fluorine atom or a chlorine atom, A represents a halogen atom, X represents an oxygen atom or a sulfur atom, and n denotes 2 or 3] and a compound represented by the general formula (10),

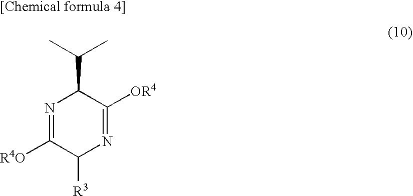

[wherein R³ represents a straight-chain alkyl group having 1 to 3 carbon atoms and R⁴ represents an alkyl group having 1 to 6 carbon atoms] to react in the presence of a base, and a step of subjecting the resultant product to acidolysis, then further protecting a nitrogen atom with a t-butoxycarbonyl group, reducing, and deprotecting the nitrogen atom.
6) A method of treating or preventing a demyelinating disease, the method comprising administrating the amino alcohol derivative according to any one of 1) to 5), or a pharmaceutically acceptable salt or hydrate thereof.
7) Use of the amino alcohol derivative according to any one of claims 1 to 5, or a pharmaceutically acceptable salt or hydrate thereof, for manufacturing a therapeutic agent or prophylactic agent for a demyelinating disease.

### ADVANTAGE OF THE INVENTION

The invention enables to provide a therapeutic agent or prophylactic agent for demyelinating diseases (multiple sclerosis, acute disseminated encephalomyelitis, adreno-leukodystrophy and adrenomyeloneuropathy, Leber hereditary optic atrophy, human T lymphotropic viral myelopathy and the like), which shows fewer side effects.

### BRIEF DESCRIPTION OF THE DRAWINGS

FIG. 1 A graph showing onset inhibitory effect of a compound 27 in a mouse EAE model: change in the symptom score (average±standard error)

FIG. 2 A graph showing onset inhibitory effect of a compound 27 in a mouse EAE model: total score (*: Shirley's multiple test, p<0.05)

### BEST MODE FOR CARRYING OUT THE INVENTION

In the present invention, the straight-chain alkyl group having 1 to 3 carbon atoms of R¹ and R³ is a methyl group, an ethyl group, or an n-propyl group.

From the perspective of obtaining high safety, R¹ is preferably an ethyl group, a propyl group, or a trifluoromethyl group, and more preferably is a trifluoromethyl group. Furthermore, R³ is preferably a methyl group, and n is preferably 3.

Furthermore, the configuration of R³ is preferably a configuration produced as the principal product via the below-described synthesis route B (using the compound (10)).

In the present invention, examples of pharmaceutically acceptable salts include acid addition salts such as hydrochloride salts, hydrobromic acid salts, acetic acid salts, trifluoroacetic acid salts, methanesulfonic acid salts, citric acid salts, or tartaric acid salts.

The active ingredient of the therapeutic agent or prophylactic agent according to the present invention represented by the general formula (1) can be produced, for example, via the synthesis route A shown below.

### <Synthesis Route A>

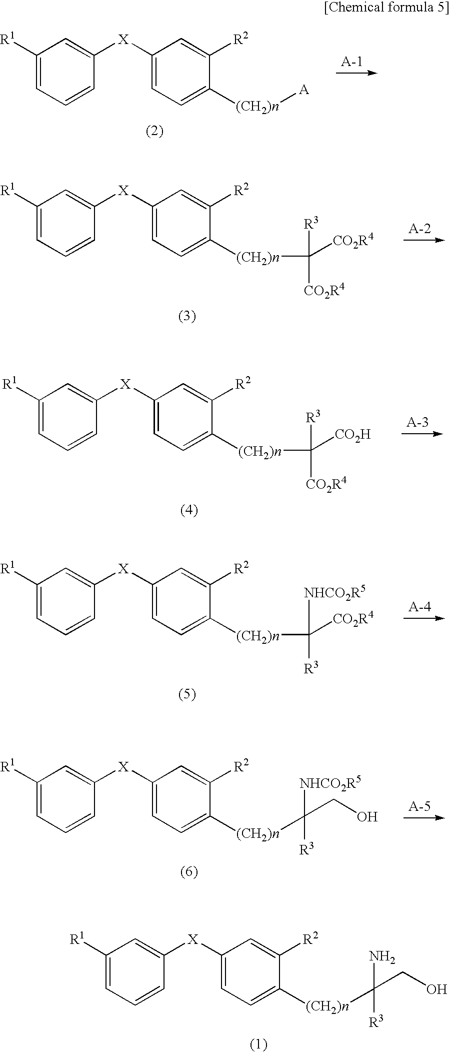

In the synthesis route A, the compound represented by the general formula (3),

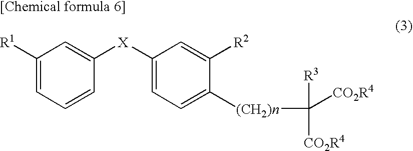

[wherein R¹, R², R³, R⁴, X, and n are as described above], can be produced by allowing a compound represented by the general formula (2),

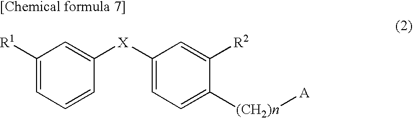

[wherein R¹, R², A, X, and n are as described above], and a compound represented by the general formula (7),

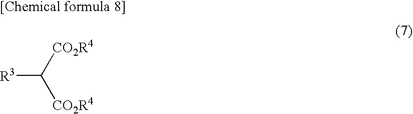

[wherein R³ and R⁴ are as described above] to act in the presence of a base (step A-1).

The reaction can be carried out using methanol, ethanol, 1,4-dioxane, dimethylsulfoxide (DMSO), N,N-dimethylformamide (DMF), tetrahydrofuran (THF) or the like as a reaction solvent, in the presence of an inorganic base such as sodium hydride, potassium hydride, sodium methoxide, sodium ethoxide, sodium t-butoxide, potassium methoxide, potassium ethoxide, potassium t-butoxide, or potassium carbonate, at 0° C. to reflux temperature as the reaction temperature, and preferably at 80° C. to 100° C.

In the synthesis route A, the compound represented by the general formula (4),

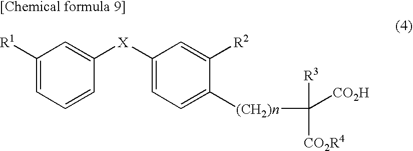

[wherein R¹, R², R³, R⁴, X, and n are as described above], can be produced by hydrolyzing the compound represented by the general formula (3) (step A-2).

The reaction can be carried out in the presence of a base such as aqueous sodium hydroxide, aqueous potassium hydroxide, or aqueous lithium hydroxide, using methanol, ethanol, 1,4-dioxane, DMF, DMSO, THF or the like as a reaction solvent, at a reaction temperature of 0° C. to reflux temperature. Preferably, the reaction is carried out using potassium hydroxide as the base, in an ethanol solvent, by reacting at 50° C.

Although the compound according to the present invention is preferably a specific optically-active substance, when the optical resolution is carried out is not especially limited. At this stage, optical resolution may be carried out by HPLC using a chiral column, whereby the desired compound having a chiral center can be obtained.

In the synthesis route A, the compound represented by the general formula (5),

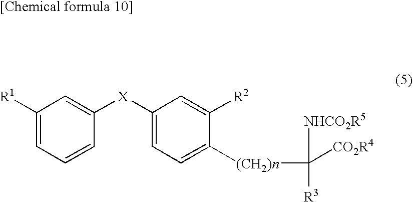

[wherein R⁵ represents an alkyl group having 1 to 6 carbon atoms, and R¹, R², R³, R⁴, X, and n are as described above], can be produced by subjecting the compound represented by the general formula (4) to Curtius rearrangement (step A-3).

In the reaction, typical methods for converting a carboxyl group into a carbamate may be employed. For example, a method which combines, for example, chloroethyl carbonate and NaN₃, or oxalyl chloride and NaN₃, or a method which uses only diphenylphosphoryl azide (DPPA) may be utilized. The reaction is preferably carried out by, after heating diphenylphosphoryl azide to reflux in the presence of an organic base, such as triethylamine, in benzene or toluene solvent, charging the resultant product with an alcohol represented by the general formula (8),

R⁵OH (8)

[herein R⁵ is as described above], and continuing to heat the resultant solution under stirring, or, after removing the solvent used in the above reaction, such as benzene or toluene, by evaporation, by heating to reflux using the alcohol represented by the general formula (8) as a reaction solvent.

At this stage, optical resolution may be carried out by HPLC using a chiral column, whereby the desired compound having a chiral center can be obtained.

In the synthesis route A, the compound represented by the general formula (6),

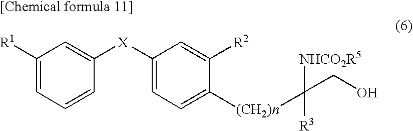

[wherein R¹, R², R³, R⁵, X, and n are as described above], can be produced by reducing the compound represented by the general formula (5) (step A-4).

The reaction can be carried out using borane, an alkyl borane derivative like 9-borabicyclo[3.3.1]nonane (9-BBN), or a metal hydride complex compound, such as diisobutylaluminum hydride ((iBu)₂AlH), sodium borohydride (NaBH₄), lithium borohydride (LiBH₄), and lithium aluminum hydride (LiAlH₄), preferably LiBH₄, using THF, 1,4-dioxane, ethanol, or methanol as a reaction solvent, at a temperature of 0° C. to reflux temperature, and preferably at room temperature.

Furthermore, at this stage also, optical resolution may be carried out by HPLC using a chiral column, whereby the desired compound having a chiral center can be obtained.

In the synthesis route A, the compound represented by the general formula (1) can be produced by subjecting the compound represented by the general formula (6) to acidolysis or hydrolysis (step A-5).

The reaction can be carried out at room temperature to reflux temperatures in an inorganic acid or organic acid, such as hydrochloric acid, hydrobromic acid, methanesulfonic acid, acetic acid, and trifluoroacetic acid, or at room temperature to reflux temperature by adding an organic solvent such as methanol, ethanol, THF, or 1,4-dioxane to an inorganic acid or organic acid, such as hydrochloric acid, hydrobromic acid, methanesulfonic acid, acetic acid, and trifluoroacetic acid. The reaction may also be carried out in the presence of a base such as aqueous sodium hydroxide, aqueous potassium hydroxide, and aqueous lithium hydroxide, using methanol, ethanol, THF, 1,4-dioxane, DMSO, or DMF as a reaction solvent, at a temperature of 0° C. to reflux temperature, and preferably 80 to 100° C.

In the synthesis route A, among the compounds represented by the general formula (5), compounds in which R⁵ represents a t-butyl group, specifically, a compound represented by the general formula (5a),

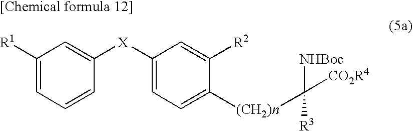

[wherein Boc represents a t-butoxycarbonyl group, and R¹, R², R³, R⁴, X, and n are as described above], and among the compounds represented by the general formula (6) in the synthesis route A, compounds in which R⁵ represents a t-butyl group, specifically, a compound represented by the general formula (6a),

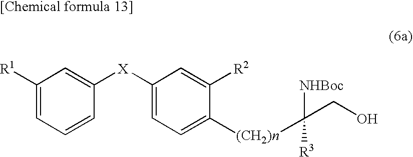

[wherein R¹, R², R³, X, Boc, and n are as described above], can be produced by the synthesis route B.

### <Synthesis Route B>

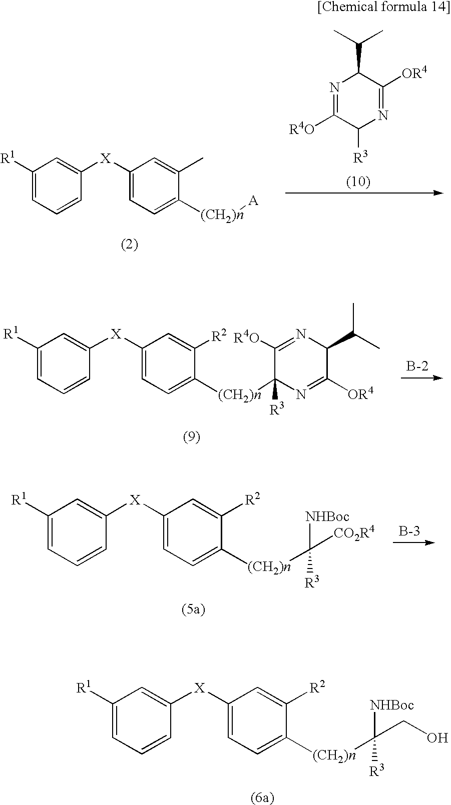

In the synthesis route B, the compound represented by the general formula (9),

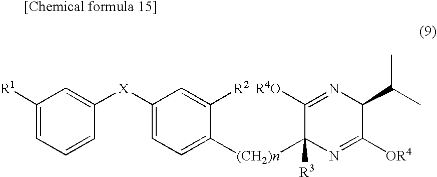

[wherein R¹, R², R³, R⁴, X, and n are as described above], can be produced by allowing a compound represented by the general formula (2) and a compound represented by the general formula (10),

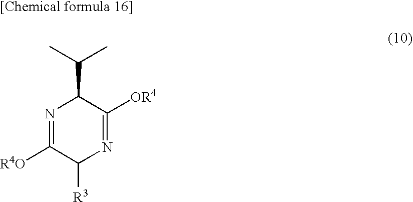

[wherein R³ and R⁴ are as described above] to react in the presence of a base (step B-1).

The reaction can be carried out using a reaction solvent such as 1,4-dioxane, THF, and ether, using a base such as n-butyllithium or lithium diisopropyl amide, preferably n-butyllithium, and treating a compound represented by the general formula (10) at −78° C., then allowing a compound represented by general formula (2) to react at −78° C., and reacting while gradually increasing the temperature to room temperature.

In the synthesis route B, the compound represented by the general formula (5a) can be produced by subjecting a compound represented by the general formula (9) to acidolysis, and then protecting the nitrogen atom with a t-butoxycarbonyl group (Boc group) (step B-2).

In the reaction, an amino ester can be obtained using methanol, ethanol, THF, 1,4-dioxane, or ethyl acetate in which hydrochloric acid is dissolved, and preferably 1,4-dioxane containing hydrochloric acid, by reacting at reflux temperature, then neutralizing with a base. Furthermore, it is preferred to allowing it to react with Boc₂O at 0° C. to room temperature using ethyl acetate, THF, DMF, 1,4-dioxane, methylene chloride, chloroform, methanol, ethanol, acetonitrile or the like as a solvent.

In the synthesis route B, the compound represented by the general formula (6a) can be produced by reducing a compound represented by the general formula (5a) (step B-3).

The reaction can be carried out using borane, an alkyl borane derivative like 9-BBN, or a metal hydride complex compound, such as (iBu)₂AlH, NaBH₄, LiBH₄, and LiAlH₄, preferably LiBH₄, using THF, 1,4-dioxane, ethanol, or methanol as a reaction solvent, at a temperature of 0° C. to reflux temperature, and preferably at room temperature.

It is noted that concerning the synthesis method of the compound represented by the general formula (2), the compound may be produced by the methods described in the respective pamphlets of WO 03029184, WO 03029205, WO 04026817, WO 04074297, and WO 050444780.

The therapeutic agent or prophylactic agent for a demyelinating disease of the invention which comprises a compound obtained in this manner as the active agent is orally or parenterally administered systemically or topically. Dosage form of the compound can be changed in response to the properties of the compound, and it is possible to be prepared as an oral preparation or a parenteral preparation. That is, granules, powders, tablets, capsules, syrups, suppositories, suspensions, solutions and the like can be prepared by mixing the active ingredient with physiologically acceptable carriers, fillers, binders, diluents and the like.

As the demyelinating disease according to the invention, multiple sclerosis, acute disseminated encephalomyelitis, adreno-leukodystrophy and adrenomyeloneuropathy, Leber hereditary optic atrophy, human T lymphotropic viral myelopathy and the like can be cited, and the therapeutic agent or prophylactic agent according to the invention is particularly useful for multiple sclerosis (Merck Manual 17th edition).

As the clinical dose, though it changes depending on the body weight, age and the condition to be treated, it is generally from 0.01 to 100 mg, preferably from 0.1 to 5 mg, per one person as the amount per once, and from 1 to 3 times per day is convenient.

In addition, the therapeutic agent or prophylactic agent for a demyelinating disease of the invention can be used in combination with one or more of other therapeutic agent or prophylactic agent for a demyelinating disease. As such a therapeutic agent or prophylactic agent for a demyelinating disease, a steroid preparation, interferon β1a, interferon β1b, glatiramer acetate, IV immunoglobulin G, mitoxantrone, cladribine, cyclophosphamide, zathioprine, methotrexate, natalizumab and an anticonvulsant can be cited. In addition, a selective potassium channel inhibitor, a thiazolidine-dione derivative, a choline esterase inhibitor, a VLA-4 inhibitor, a CCR1 inhibitor, a PDE4 inhibitor, a cathepsin S inhibitor, an mTOR inhibitor and the like can also be used.

### EXAMPLES

Next, the present invention will be described with the following specific examples. However, the present invention is not limited by these examples.

Furthermore, as the intermediates and the like represented by the general formula (2), the compounds in the pamphlets of WO 03029184, WO 03029205, WO 04026817, WO 04074297, and WO 050444780 may be utilized. Furthermore, (5S)-3,6-diethoxy-5-isopropyl-2-methyl-2,5-dihydropyrazine, (5S)-3,6-dimethoxy-5-isopropyl-2-methyl-2,5-dihydropyrazine, and (5S)-2-allyl-3,6-diethoxy-5-isopropyl-2,5-dihydropyrazine may be synthesized according to Ulrich Shollkopf et. al, Synthesis 969 (1981) and Chunrong Ma et. al., J. Org. Chem., 66, 4525 (2001). Intermediates and the like which were newly synthesized based on the experiment procedures described in these reference documents will now be described as the following reference examples.

### Reference Example 1

### 2-Fluoro-4-(3-trifluoromethylphenylthio)benzaldehyde

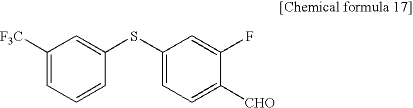

Under an argon atmosphere, ethyldiisopropylamine (7.0 mL), tris(dibenzylideneacetone)dipalladium(0) chloroform adduct (518 mg), xantphos (578 mg), and 3-trifluoromethylthiophenol (3.56 g) were added at room temperature into a solution of 4-bromo-2-fluorobenzaldehyde (4.06 g) in 1,4-dioxane (42 mL), and the resultant solution was heated to reflux for 5 hours. To the reaction solution added water, extracted with ethyl acetate, washed with water and saturated brine in that order, and then dried over anhydrous sodium sulfate. The solvent was evaporated, and the resultant residue was purified by silica gel column chromatography (hexane:ethyl acetate=30:1) to obtain the target product (4.08 g) as a colorless oil.

¹H-NMR (CDCl₃, 400 MHz): δ 6.86 (1H, dd, J=10, 1.8 Hz), 7.02 (1H, dd, J=7.9, 1.8 Hz), 7.58 (1H, t, J=7.9 Hz), 7.68-7.73 (2H, m), 7.76 (1H, t, J=7.9 Hz), 7.80 (1H, s), 10.26 (1H, s).

EIMS (+): 300 [M]⁺.

### Reference Example 2

### 2-Chloro-4-(3-chlorophenylthio)benzaldehyde

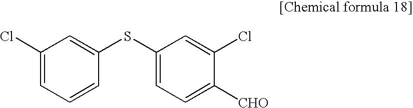

3-Chlorobenzenethiol and 2-chloro-4-fluorobenzaldehyde were reacted according to the same experiment procedures as in Reference Example 1 of the pamphlet of WO 03029205 to obtain the target product as a colorless oil.

¹H-NMR (CDCl₃, 400 MHz): δ 7.11 (1H, dd, J=9.2, 1.8 Hz), 7.17 (1H, d, J=1.8 Hz), 7.36-7.44 (3H, m), 7.52 (1H, t, J=1.8 Hz), 7.80 (1H, d, J=7.9 Hz), 10.37 (1H, s).

EIMS (+): 282 [M]⁺.

### Reference Example 3

### 2-Chloro-4-(3-methylphenoxy)benzaldehyde

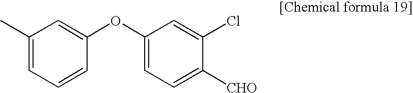

m-Cresol and 2-chloro-4-fluorobenzaldehyde were reacted according to the same experiment procedures as in Reference Example 1 of the pamphlet of WO 03029184 to obtain the target product as a colorless powder.

¹H-NMR (CDCl₃, 400 MHz): δ 2.38 (3H, s), 6.87-6.96 (4H, m), 7.07 (1H, d, J=7.3 Hz), 7.31 (1H, t, J=7.6 Hz), 7.90 (1H, d, J=8.6 Hz), 10.36 (1H, s).

EIMS (+): 246 [M]⁺.

### Reference Example 4

### 2-Chloro-4-(3-ethylphenylthio)benzaldehyde

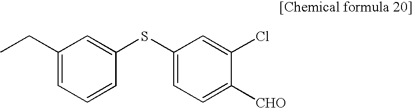

3-Ethylbenzenethiol and 2-chloro-4-fluorobenzaldehyde were reacted according to the same experiment procedures as in Reference Example 1 of the pamphlet of WO 03029205 to obtain the target product as a colorless oil.

¹H-NMR (CDCl₃, 400 MHz): δ 1.26 (3H, t, J=7.3 Hz), 2.68 (2H, q, J=7.3 Hz), 7.04-7.11 (2H, m), 7.28-7.40 (4H, m), 7.76 (1H, d, J=8.6 Hz), 10.35 (1H, s).

EIMS (+): 276 [M]⁺.

### Reference Example 5

### 2-Chloro-4-(3-propylphenoxy)benzaldehyde

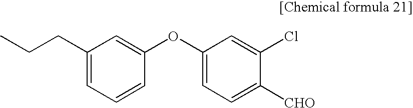

3-Propylphenol and 2-chloro-4-fluorobenzaldehyde were reacted according to the same experiment procedures as in Reference Example 1 of the pamphlet of WO 03029184 to obtain the target product as a pale brown oil.

¹H-NMR (CDCl₃, 400 MHz): δ 0.95 (3H, t, J=7.3 Hz), 1.62-1.68 (2H, m), 2.61 (2H, t, J=7.3 Hz), 6.89-6.94 (3H, m), 6.96 (1H, d, J=2.1 Hz), 7.08 (1H, d, J=7.9 Hz), 7.31-7.35 (1H, m), 7.90 (1H, d, J=8.9 Hz), 10.36 (1H, d, J=0.6 Hz).

EIMS (+): 274 [M]⁺.

### Reference Example 6

### [2-Chloro-4-(3-ethylphenylthio)phenyl]acetaldehyde

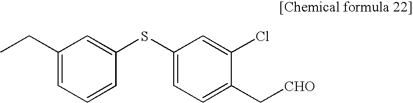

The compound of Reference Example 4 was reacted according to the same experiment procedures as in Reference Example 326 of the pamphlet of WO 04074297 to obtain the target product as a pale yellow oil.

### Reference Example 7

### Ethyl 3-[2-chloro-4-(3-ethylphenylthio)phenyl]acrylate

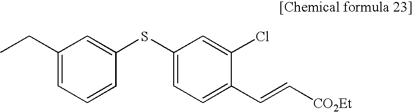

The compound of Reference Example 4 was reacted according to the same experiment procedures as in Reference Example 10 of the pamphlet of WO 03029205 to obtain the target product as a pale yellow oil.

EIMS (+): 346 [M]⁺.

### Reference Example 8

### 3-[2-Chloro-4-(3-ethylphenylthio)phenyl]propan-1-ol

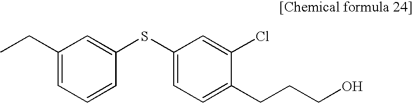

The compound of Reference Example 7 was reacted according to the same experiment procedures as in Reference Example 19 of the pamphlet of WO 03029205, and the resultant product was then reduced according to the same experiment procedures as in Reference Example 35 of the pamphlet of WO 03029205, to obtain the target product as a colorless oil.

¹H-NMR (CDCl₃, 400 MHz,): δ 1.22 (3H, t, J=7.3 Hz), 1.84-1.90 (2H, m), 2.62 (2H, q, J=7.6 Hz), 2.78-2.82 (2H, m), 3.69 (2H, t, J=6.1 Hz), 7.10-7.18 (4H, m), 7.23-7.29 (3H, m).

### Reference Example 9

### 3-[2-Chloro-4-(3-propylphenoxy)phenyl]propan-1-ol

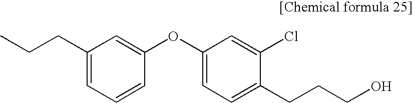

The compound of Reference Example 5 was successively reacted according to the same procedures as in Reference Example 7 and then Reference Example 8 to obtain the target product as a colorless oil.

¹H-NMR (CDCl₃, 400 MHz,): δ 0.94 (3H, t, J=7.3 Hz), 1.37 (1H, br s), 1.58-1.68 (2H, m), 1.85-1.92 (2H, m), 2.57 (2H, t, J=7.6 Hz), 2.80 (2H, t, J=7.6 Hz), 3.70 (2H, dt, J=6.1, 4.6 Hz), 6.80-6.85 (3H, m), 6.95 (1H, d, J=7.9 Hz), 7.00 (1H, d, J=2.8 Hz), 7.17 (1H, d, J=8.3 Hz), 7.24 (1H, t, J=7.9 Hz).

EIMS (+): 304 [M]⁺.

### Reference Example 10

### 3-[2-Fluoro-4-(3-trifluoromethylphenylthio)phenyl]propan-1-ol

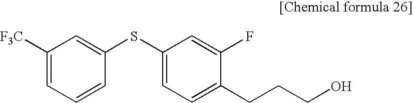

The compound of Reference Example 1 was successively reacted according to the same procedures as in Reference Example 7 and then Reference Example 8 to obtain the target product as a colorless oil.

¹H-NMR (CDCl₃, 400 MHz): δ 1.88 (2H, tt, J=6.7, 6.1 Hz), 2.75 (2H, t, J=6.7 Hz), 3.69 (2H, t, J=6.1 Hz), 7.05 (1H, dd, J=10, 1.8 Hz), 7.10 (1H, dd, J=7.9, 1.8 Hz), 7.20 (1H, t, J=7.9 Hz), 7.38-7.51 (3H, m), 7.55 (1H, s).

### Reference Example 11

### 3-[2-Chloro-4-(3-chlorophenylthio)phenyl]propan-1-ol

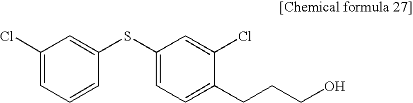

The compound of Reference Example 2 was successively reacted according to the same procedures as in Reference Example 7 and then Reference Example 8 to obtain the target product as a colorless oil.

¹H-NMR (CDCl₃, 400 MHz): δ 1.33 (1H, br s), 1.83-1.95 (2H, m), 2.81-2.85 (2H, m), 3.70 (2H, br s), 7.15-7.23 (5H, m), 7.24-7.29 (1H, m), 7.38 (1H, d, J=1.8 Hz).

### Reference Example 12

### 3-[2-Chloro-4-(3-methylphenoxy)phenyl]propan-1-ol

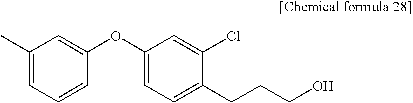

The compound of Reference Example 3 was successively reacted according to the same procedures as in Reference Example 7 and then Reference Example 8 to obtain the target product as a colorless oil.

¹H-NMR (CDCl₃, 400 MHz): δ 1.31 (1H, brs), 1.87-1.90 (2H, m), 2.34 (3H, s), 2.80 (2H, t, J=7.3 Hz), 3.70 (2H, dd, J=11.6, 6.1 Hz), 6.79-6.86 (3H, m), 6.94 (1H, d, J=7.3 Hz), 6.99 (1H, d, J=2.4 Hz), 7.18 (1H, d, J=7.9 Hz), 7.22 (1H, t, J=7.3 Hz).

EIMS (+): 276 [M]⁺.

### Reference Example 13

### 2-Chloro-4-(3-ethylphenylthio)-1-(2-iodoethyl)benzene

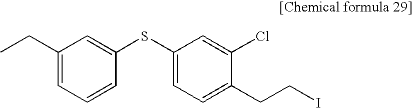

The compound of Reference Example 6 was reacted according to the same experiment procedures as in Reference Example 327 of the pamphlet of WO 04074297 to obtain the target product as a colorless oil.

¹H-NMR (CDCl₃, 400 MHz): δ 1.22 (3H, t, J=7.3 Hz), 2.63 (2H, q, J=7.3 Hz), 3.23-3.28 (2H, m), 3.32-3.35 (2H, m), 7.09-7.29 (7H, m).

EIMS (+): 402 [M]⁺.

### Reference Example 14

### 2-Chloro-4-(3-ethylphenylthio)-1-(3-iodopropyl)benzene

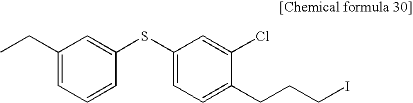

The compound of Reference Example 8 was reacted according to the same experiment procedures as in Reference Example 164 of the pamphlet of WO 03029184 to obtain the target product as a colorless oil.

¹H-NMR (CDCl₃, 400 MHz): δ 1.22 (3H, t, J=7.3 Hz), 2.12 (2H, quintet, J=7.3 Hz), 2.63 (2H, q, J=7.3 Hz), 2.81 (2H, t, J=7.3 Hz), 3.19 (2H, t, J=7.3 Hz), 7.09-7.19 (4H, m), 7.24-7.28 (3H, m).

EIMS (+): 416 [M]⁺.

### Reference Example 15

### 2-Chloro-1-(3-iodopropyl)-4-(3-propylphenoxy)benzene

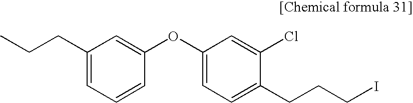

The compound of Reference Example 9 was reacted according to the same experiment procedures as in Reference Example 164 of the pamphlet of WO 03029184 to obtain the target product as a pale yellow oil.

¹H-NMR (CDCl₃, 400 MHz): δ 0.94 (3H, t, J=7.3 Hz), 1.60-1.68 (2H, m), 2.10-2.17 (2H, m), 2.57 (2H, t, J=7.6 Hz), 2.81 (2H, t, J=7.6 Hz), 3.21 (2H, t, J=7.0 Hz), 6.80-6.85 (3H, m), 6.96 (1H, d, J=7.9 Hz), 6.99 (1H, d, J=2.4 Hz), 7.19 (1H, d, J=8.3 Hz), 7.25 (1H, t, J=7.9 Hz).

EIMS (+): 414 [M]⁺.

### Reference Example 16

### 2-Fluoro-1-(3-iodopropyl)-4-(3-trifluoromethylphenylthio)benzene

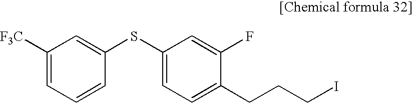

The compound of Reference Example 10 was reacted according to the same experiment procedures as in Reference Example 164 of the pamphlet of WO 03029184 to obtain the target product as a colorless oil.

¹H-NMR (CDCl₃, 400 MHz): δ 2.13 (2H, quintet, J=7.3 Hz), 2.76 (2H, t, J=7.3 Hz), 3.18 (2H, t, J=6.7 Hz), 7.03 (1H, dd, J=10, 1.8 Hz), 7.09 (1H, dd, J=7.9, 1.8 Hz), 7.20 (1H, t, J=7.9 Hz), 7.39-7.52 (3H, m), 7.57 (1H, s).

EIMS (+): 404 [M]⁺.

### Reference Example 17

### 2-Chloro-4-(3-chlorophenylthio)-1-(3-iodopropyl)benzene

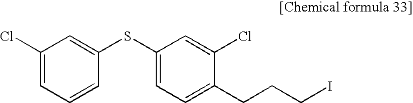

The compound of Reference Example 11 was reacted according to the same experiment procedures as in Reference Example 164 of the pamphlet of WO 03029184 to obtain the target product as a colorless oil.

¹H-NMR (CDCl₃, 400 MHz): δ 2.14 (2H, tt, J=7.3, 6.7 Hz), 2.84 (2H, t, J=7.3 Hz), 3.20 (2H, t, J=6.7 Hz), 7.16-7.25 (5H, m), 7.28 (1H, t, J=1.8 Hz), 7.36 (1H, d, J=1.8 Hz).

EIMS (+): 422 [M]⁺.

### Reference Example 18

### 2-Chloro-1-(3-iodopropyl)-4-(3-methylphenoxy)benzene

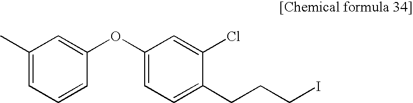

The compound of Reference Example 12 was reacted according to the same experiment procedures as in Reference Example 164 of the pamphlet of WO 03029184 to obtain the target product as a yellow oil.

¹H-NMR (CDCl₃, 400 MHz): δ 2.13 (2H, quint, J=7.3 Hz), 2.34 (3H, s), 2.81 (2H, t, J=7.3 Hz), 3.21 (2H, t, J=7.3 Hz), 6.81-6.84 (3H, m), 6.95 (1H, d, J=7.9 Hz), 6.99 (1H, d, J=2.4 Hz), 7.18 (1H, d, J=7.9 Hz), 7.23 (1H, t, J=7.9 Hz).

EIMS (+): 386 [M]⁺.

### Example 1

### (2R,5S)-2-[2-chloro-4-(3-trifluoromethylphenoxy)phenyl]propyl-3,6-diethoxy-5-isopropyl-2-methyl-2,5-dihydropyrazine

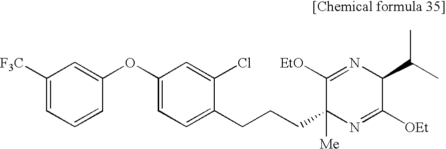

Under an argon atmosphere, a solution of n-butyllithium in hexane (1.54 mol/L, 3.59 mL) was added at −78° C. into a solution of (5S)-3,6-diethoxy-5-isopropyl-2-methyl-2,5-dihydropyrazine (905 mg) in THF (16 mL), and the resultant solution was stirred at −78° C. for 30 minutes. Next, A solution of 2-chloro-1-(3-iodopropyl)-4-(3-trifluoromethylphenoxy)benzene (2.47 g) in THF (4 mL) was added to the reaction mixture, and the resultant solution was stirred at −78° C. for 30 minutes and then at 0° C. for 1 hour. To the reaction solution was added water, extracted with ethyl acetate, washed with water and saturated brine in that order, and then dried over anhydrous sodium sulfate. The solvent was evaporated, and the resultant residue was purified by silica gel column chromatography (hexane:ethyl acetate=60:1) to obtain the target product (1.59 g) as a colorless oil.

¹H-NMR (CDCl₃, 400 MHz): δ 0.70 (3H, d, J=6.7 Hz), 1.05 (3H, d, J=6.7 Hz), 1.18-1.50 (9H, m), 1.32 (3H, s), 1.86-1.97 (1H, m), 2.21-2.30 (1H, m), 2.65 (2H, t, J=7.6 Hz), 3.90 (1H, d, J=2.1 Hz), 3.97-4.21 (4H, m), 6.84 (1H, dd, J=7.9, 2.4 Hz), 7.00 (1H, d, J=2.4 Hz), 7.15 (2H, d, J=7.9 Hz), 7.24 (1H, br s), 7.36 (1H, d, J=7.9 Hz), 7.44 (1H, t, J=7.9 Hz).

### Example 2

### (2R,5S)-2-[2-chloro-4-(3-trifluoromethylphenylthio)phenyl]propyl-3,6-diethoxy-5-isopropyl-2-methyl-2,5-dihydropyrazine

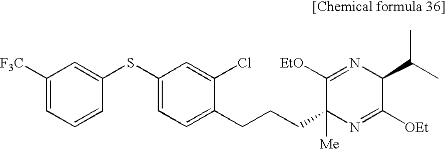

(5S)-3,6-diethoxy-5-isopropyl-2-methyl-2,5-dihydropyrazine and 2-chloro-1-(3-iodopropyl)-4-(3-trifluoromethylphenylthio)benzene were reacted in the same manner as in Example 1 to obtain the target product as a colorless oil.

¹H-NMR (CDCl₃, 400 MHz): δ 0.63 (3H, d, J=6.7 Hz), 1.07 (3H, d, J=6.7 Hz), 1.18-1.29 (10H, m), 1.34-1.66 (2H, m), 1.79-1.91 (1H, m), 2.25-2.33 (1H, m), 2.70 (2H, t, J=7.6 Hz), 3.85 (1H, br s), 3.99-4.23 (4H, m), 7.16 (2H, d, J=7.9 Hz), 7.20 (1H, dd, J=7.9, 1.8 Hz), 7.36-7.42 (3H, m), 7.44-7.50 (1H, m), 7.52 (1H, br s).

### Example 3

### (2R,5S)-2-[2-chloro-4-(3-trifluoromethylphenoxy)phenyl]ethyl-3,6-diethoxy-5-isopropyl-2-methyl-2,5-dihydropyrazine

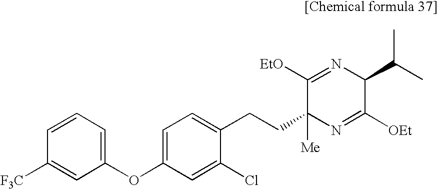

(5S)-3,6-diethoxy-5-isopropyl-2-methyl-2,5-dihydropyrazine and 2-chloro-1-(2-iodoethyl)-4-(3-trifluoromethylphenoxy)benzene were reacted in the same manner as in Example 1 to obtain the target product as a colorless oil.

¹H-NMR (CDCl₃, 400 MHz): δ 0.72 (3H, d, J=6.7 Hz), 1.08 (3H, d, J=6.7 Hz), 1.29 (6H, t, J=7.3 Hz), 1.36 (3H, s), 1.74-1.82 (1H, m), 2.13-2.20 (1H, m), 2.25-2.32 (1H, m), 2.39-2.56 (2H, m), 3.95 (1H, d, J=3.1 Hz), 4.02-4.22 (4H, m), 6.83 (1H, dd, J=8.6, 2.4 Hz), 6.99 (1H, d, J=2.4 Hz), 7.12-7.15 (2H, m), 7.23 (1H, br s), 7.35 (1H, d, J=7.8 Hz), 7.44 (1H, t, J=7.8 Hz).

EIMS (+): 524 [M]⁺.

### Example 4

### (2R,5S)-2-[2-chloro-4-(3-trifluoromethylphenylthio)phenyl]ethyl-3,6-diethoxy-5-isopropyl-2-methyl-2,5-dihydropyrazine

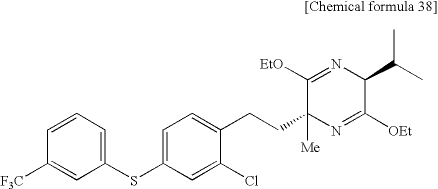

(5S)-3,6-diethoxy-5-isopropyl-2-methyl-2,5-dihydropyrazine and 2-chloro-1-(2-iodoethyl)-4-(3-trifluoromethylphenylthio)benzene were reacted in the same manner as in Example 1 to obtain the target product as a colorless oil.

¹H-NMR (CDCl₃, 400 MHz): δ 0.72 (3H, d, J=6.7 Hz), 1.08 (3H, d, J=6.7 Hz), 1.28 (6H, t, J=7.3 Hz), 1.35 (3H, s), 1.68-1.90 (1H, m), 2.10-2.19 (1H, m), 2.38-2.57 (1H, m), 3.95 (1H, d, J=3.1 Hz), 4.02-4.22 (4H, m), 7.13 (1H, d, J=7.9 Hz), 7.18 (1H, dd, J=7.9, 2.4 Hz), 7.35-7.42 (3H, m), 7.43-7.48 (1H, m), 7.54 (1H, br s).

### Example 5

### (2R,5S)-2-[2-chloro-4-(3-ethylphenylthio)phenyl]ethyl-3,6-diethoxy-5-isopropyl-2-methyl-2,5-dihydropyrazine

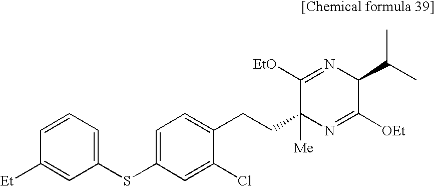

(5S)-3,6-diethoxy-5-isopropyl-2-methyl-2,5-dihydropyrazine and the compound of Reference Example 13 were reacted in the same manner as in Example 1 to obtain the target product as a colorless oil.

¹H-NMR (CDCl₃, 400 MHz): δ 0.72 (3H, d, J=6.7 Hz), 1.07 (3H, d, J=6.7 Hz), 1.21 (3H, t, J=7.3 Hz), 1.28 (3H, t, J=7.3 Hz), 1.29 (3H, t, J=7.3 Hz), 1.34 (3H, s), 1.70-1.79 (1H, m), 2.09-2.16 (1H, m), 2.24-2.32 (1H, m), 2.35-2.52 (2H, m), 2.61 (2H, q, J=7.3 Hz), 3.95 (1H, d, J=3.1 Hz), 4.03-4.20 (4H, m), 7.04-7.15 (4H, m), 7.21-7.26 (3H, m).

ESIMS (+): 501 [M+H]⁺.

### Example 6

### (2R,5S)-2-[2-chloro-4-(3-methylphenoxy)phenyl]propyl-3,6-dimethoxy-5-isopropyl-2-methyl-2,5-dihydropyrazine

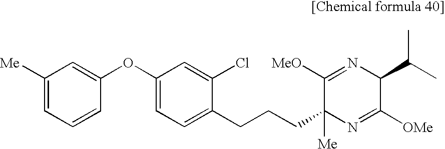

(5S)-3,6-dimethoxy-5-isopropyl-2-methyl-2,5-dihydropyrazine and the compound of Reference Example 18 were reacted in the same manner as in Example 1 to obtain the target product as a colorless oil.

¹H-NMR (CDCl₃, 400 MHz): δ 0.68 (3H, d, J=6.7 Hz), 1.07 (3H, d, J=6.7 Hz), 1.33 (3H, s), 1.36-1.43 (1H, m), 1.55-1.62 (1H, m), 1.86-1.92 (1H, m), 2.24-2.26 (1H, m), 2.34 (3H, s), 2.62 (2H, t, J=7.9 Hz), 3.65 (3H, s), 3.66 (3H, s), 3.94 (1H, d, J=3.7 Hz), 6.79-6.82 (3H, m), 6.93 (1H, d, J=7.3 Hz), 6.96 (1H, d, J=2.4 Hz), 7.09 (1H, d, J=7.9 Hz), 7.22 (1H, t, J=7.9 Hz).

EIMS (+): 456 [M]⁺.

### Example 7

### (2R,5S)-2-[2-chloro-4-(3-ethylphenylthio)phenyl]propyl-3,6-diethoxy-5-isopropyl-2-methyl-2,5-dihydropyrazine

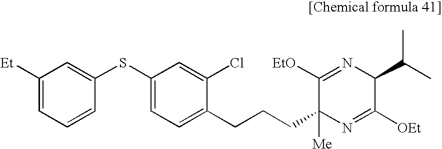

(5S)-3,6-diethoxy-5-isopropyl-2-methyl-2,5-dihydropyrazine and the compound of Reference Example 14 were reacted in the same manner as in Example 1 to obtain the target product as a colorless oil.

¹H-NMR (CDCl₃, 400 MHz): δ 0.68 (3H, d, J=6.7 Hz), 1.04 (3H, d, J=6.7 Hz), 1.20-1.26 (9H, m), 1.31 (3H, s), 1.36-1.43 (1H, m), 1.50-1.57 (1H, m), 1.85-1.92 (1H, m), 2.21-2.28 (1H, m), 2.60-2.65 (4H, m), 3.88 (1H, d, J=3.7 Hz), 4.00-4.16 (4H, m), 7.06-7.16 (4H, m), 7.22-7.27 (3H, m).

ESIMS (+): 515 [M+H]⁺.

### Example 8

### (2R,5S)-2-[2-chloro-4-(3-chlorophenylthio)phenyl]propyl-3,6-diethoxy-5-isopropyl-2-methyl-2,5-dihydropyrazine

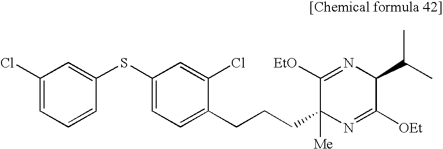

(5S)-3,6-diethoxy-5-isopropyl-2-methyl-2,5-dihydropyrazine and the compound of Reference Example 17 were reacted in the same manner as in Example 1 to obtain the target product as a colorless oil.

¹H-NMR (CDCl₃, 400 MHz): δ 0.69 (3H, d, J=6.7 Hz), 1.08 (3H, d, J=6.7 Hz), 1.18-1.29 (7H, m), 1.31 (3H, s), 1.34-1.47 (1H, m), 1.50-1.63 (1H, m), 1.85-1.95 (1H, m), 2.20-2.30 (1H, m), 2.65 (2H, t, J=7.6 Hz), 3.89 (1H, d, J=3.1 Hz), 3.99-4.23 (4H, m), 7.11-7.23 (6H, m), 7.35 (1H, d, J=1.8 Hz).

ESIMS (+): 521 [M+H]⁺.

### Example 9

### (2R,5S)-2-[2-fluoro-4-(3-trifluoromethylphenylthio)phenyl]propyl-3,6-diethoxy-5-isopropyl-2-methyl-2,5-dihydropyrazine

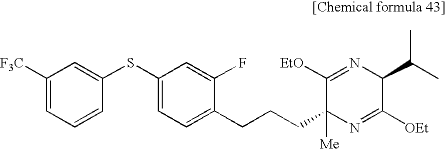

(5S)-3,6-diethoxy-5-isopropyl-2-methyl-2,5-dihydropyrazine and the compound of Reference Example 16 were reacted in the same manner as in Example 1 to obtain the target product as a colorless oil.

¹H-NMR (CDCl₃, 400 MHz): δ 0.67 (3H, d, J=6.7 Hz), 1.06 (3H, d, J=6.7 Hz), 1.18-1.29 (7H, m), 1.33 (3H, s), 1.36-1.66 (2H, m), 1.85-1.95 (1H, m), 2.23-2.33 (1H, m), 2.67 (2H, t, J=7.6 Hz), 3.89 (1H, d, J=3.1 Hz), 3.99-4.23 (4H, m), 7.02 (1H, dd, J=9.8 Hz, 1.8 Hz), 7.08 (1H, dd, J=7.9 Hz, 2.4 Hz), 7.13 (1H, t, J=7.9 Hz), 7.38-7.50 (3H, m), 7.55 (1H, s).

### Example 10

### (2S,5S)-2-allyl-2-[2-chloro-4-(3-trifluoromethylphenylthio)phenyl]propyl-3,6-diethoxy-5-isopropyl-2,5-dihydropyrazine

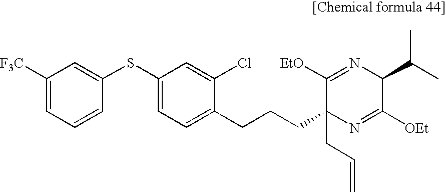

(5S)-2-allyl-3,6-diethoxy-5-isopropyl-2,5-dihydropyrazine and 2-chloro-1-(3-iodopropyl)-4-(3-trifluoromethylphenylthio)benzene were reacted in the same manner as in Example 1 to obtain the target product as a colorless oil.

¹H-NMR (CDCl₃, 400 MHz): δ 0.67 (3H, d, J=6.7 Hz), 1.05 (3H, d, J=6.7 Hz), 1.23 (3H, t, J=6.4 Hz), 1.25 (3H, t, J=6.4 Hz), 1.30-1.64 (3H, m), 1.80-1.90 (1H, m), 2.23-2.39 (2H, m), 2.53 (1H, dd, J=12.4, 7.3 Hz), 2.65 (2H, t, J=7.6 Hz), 3.83 (1H, d, J=3.1 Hz), 4.03-4.18 (4H, m), 4.92-5.04 (2H, m), 5.60-5.73 (1H, m), 7.13 (2H, d, J=7.9 Hz), 7.18 (1H, dd, J=7.9 Hz, 1.8 Hz), 7.36 (1H, d, J=1.8 Hz), 7.38-7.42 (2H, m), 7.44-7.49 (1H, m), 7.55 (1H, br s).

### Example 11

### Ethyl(R)-2-t-butoxycarbonylamino-5-[2-chloro-4-(3-trifluoromethylphenoxy)phenyl]-2-methylpentanoate

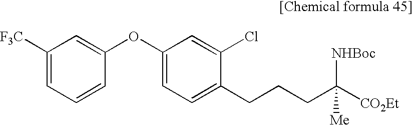

To a solution of the compound of Example 1 (1.59 g) in 1,4-dioxane (60 mL) was added 0.5 mol/L hydrochloric acid (30 mL). The resultant solution was stirred at room temperature for 1 hour, and then left to stand at room temperature overnight. The solution was concentrated, neutralized with saturated aqueous sodium hydrogen carbonate solution, and extracted with ethyl acetate. The extract was washed with water and saturated brine, and then dried over anhydrous sodium sulfate. The extract was concentrated, and the resultant residue was dissolved in acetonitrile (15 mL). To this solution was added di-tert-butoxydicarbonate (1.55 g), and the resultant solution was stirred at room temperature for 4 hours and then left to stand at room temperature overnight. To the reaction solution added water, extracted with ethyl acetate, washed with water and saturated brine in that order, and then dried over anhydrous sodium sulfate. The solvent was evaporated, and the resultant residue was purified by silica gel column chromatography (hexane:ethyl acetate=9:1) to obtain the target product (1.00 g) as a colorless oil.

¹H-NMR (CDCl₃, 400 MHz): δ 1.26 (3H, t, J=7.3 Hz), 1.43 (9H, s), 1.53 (3H, s), 1.45-1.68 (2H, m), 1.80-1.90 (1H, m), 2.12-2.30 (1H, m), 2.69 (2H, t, J=7.6 Hz), 4.16-4.24 (2H, m), 5.33 (1H, br s), 6.85 (1H, dd, J=7.9 Hz, 2.4 Hz), 7.02 (1H, d, J=2.4 Hz), 7.15 (1H, dd, J=7.9 Hz, 2.4 Hz), 7.17 (1H, d, J=7.9 Hz), 7.24 (1H, br s), 7.37 (1H, d, J=7.9 Hz), 7.45 (1H, t, J=7.9 Hz).

### Example 12

### Ethyl(R)-2-t-butoxycarbonylamino-5-[2-fluoro-4-(3-trifluoromethylphenylthio)phenyl]-2-methylpentanoate

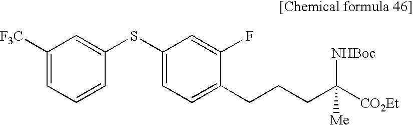

The compound of Example 9 was reacted in the same manner as in Example 11 to obtain the target product as a colorless oil.

¹H-NMR (CDCl₃, 400 MHz): δ 1.26 (3H, t, J=7.3 Hz), 1.42 (9H, s), 1.51 (3H, s), 1.45-1.68 (2H, m), 1.77-1.86 (1H, m), 2.09-2.20 (1H, m), 2.69 (2H, t, J=7.6 Hz), 4.13-4.23 (2H, m), 5.29 (1H, br s), 7.02 (1H, dd, J=9.8 Hz, 1.8 Hz), 7.08 (1H, dd, J=7.9 Hz, 2.4 Hz), 7.13 (1H, t, J=7.9 Hz), 7.38-7.50 (3H, m), 7.55 (1H, s).

### Example 13

### Ethyl(S)-2-allyl-2-t-butoxycarbonylamino-5-[2-chloro-4-(3-trifluoromethylphenylthio)phenyl]pentanoate

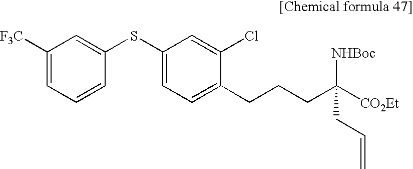

The compound of Example 10 was reacted in the same manner as in Example 11 to obtain the target product as a colorless oil.

¹H-NMR (CDCl₃, 400 MHz) δ 1.24 (3H, t, J=7.3 Hz), 1.29-1.39 (1H, m), 1.43 (9H, s), 1.60-1.70 (1H, m), 1.78-1.86 (1H, m), 2.32-2.50 (2H, m), 2.66-2.73 (2H, m), 2.99-3.10 (1H, m), 4.19 (2H, q), 5.03 (1H, d, J=3.1 Hz), 5.09 (1H, s), 5.49 (1H, br s), 5.54-5.68 (1H, m), 7.16 (1H, d, J=7.9 Hz), 7.19 (1H, dd, J=7.9, 1.8 Hz), 7.35 (1H, d, J=1.8 Hz), 7.39-7.44 (2H, m), 7.45-7.50 (1H, m), 7.54 (1H, br s).

### Example 14

### Ethyl(R)-2-t-butoxycarbonylamino-5-[2-chloro-4-(3-trifluoromethylphenylthio)phenyl]-2-propylpentanoate

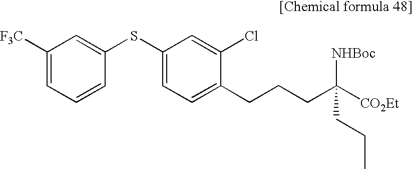

To a solution of the compound of Example 13 (400 mg) in ethyl acetate (20 mL) was added palladium, on activated carbon/ethylene diamine complex (100 mg), and the resultant solution was stirred at room temperature for 24 hours under hydrogen atmosphere. The reaction solution was filtered through Celite, and the solvent was evaporated. The resultant residue was purified by silica gel column chromatography (hexane:ethyl acetate=30:1) to obtain the target product (293 mg) as a colorless oil.

¹H-NMR (CDCl₃, 400 MHz): δ 0.91 (3H, t, J=7.3 Hz), 1.42 (9H, s), 1.15-1.77 (8H, m), 2.72 (2H, t, J=7.3 Hz), 3.63 (1H, d, J=12 Hz), 3.67 (1H, d, J=12 Hz), 4.52 (1H, br s), 7.19-7.22 (2H, m), 7.39 (1H, s), 7.40-7.50 (3H, m), 7.54 (1H, br s).

FABMS (+): 532 [M+H]⁺.

### Example 15

### (R)-2-t-butoxycarbonylamino-5-[2-chloro-4-(3-trifluoromethylphenoxy)phenyl]-2-methylpentan-1-ol

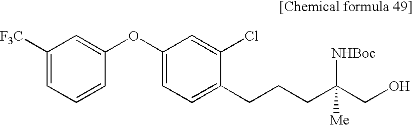

To a solution of the compound of Example 11 (1.00 g) in THF (14 mL) was added under ice cooling lithium borohydride (229 mg), and then ethanol (1.4 mL) was added dropwise. The resultant solution was then stirred for 1 hour under ice cooling. To the reaction solution was added 10% aqueous citric acid, extracted with ethyl acetate, washed with water and saturated brine in that order, and then dried over anhydrous sodium sulfate. The solvent was evaporated, and the resultant residue was purified by silica gel column chromatography (hexane:ethyl acetate=4:1) to obtain the target product (910 mg) as a colorless oil.

¹H-NMR (CDCl₃, 400 MHz): δ 1.16 (3H, s), 1.43 (9H, s), 1.53-1.74 (3H, m), 1.81-1.93 (1H, m), 2.73 (2H, t, J=7.3 Hz), 3.61 (1H, d, J=12 Hz), 3.65 (1H, d, J=12 Hz), 4.58 (1H, br s), 4.58 (1H, br s), 6.86 (1H, dd, J=7.9, 2.4 Hz), 7.03 (1H, d, J=2.4 Hz), 7.16 (1H, dd, J=7.9 Hz, 2.4 Hz), 7.21 (1H, d, J=7.9 Hz), 7.24 (1H, br s), 7.37 (1H, d, J=7.9 Hz), 7.45 (1H, t, J=7.9 Hz).

### Example 16

### (R)-2-t-butoxycarbonylamino-5-[2-chloro-4-(3-trifluoromethylphenylthio)phenyl]-2-methylpentan-1-ol

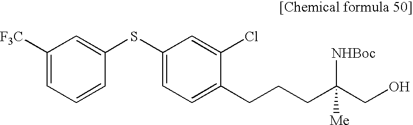

The compound of Example 2 was reacted in the same manner as in Example 11 to obtain an ester, which was then reacted in the same manner as in Example 15 to obtain the target product as a colorless oil.

¹H-NMR (CDCl₃, 400 MHz): δ 1.14 (3H, s), 1.42 (9H, s), 1.48-1.76 (4H, m), 1.81-1.90 (1H, m), 2.74 (2H, t, J=6.7 Hz), 3.61 (1H, d, J=12 Hz), 3.65 (1H, d, J=12 Hz), 4.56 (1H, br s), 4.58 (1H, br s), 7.20 (2H, d, J=1.2 Hz), 7.37-7.50 (4H, m), 7.54 (1H, br s).

Optical Rotation: [α]_{D}²⁷+14.31 (c 0.63, CHCl₃).

### Example 17

### (R)-2-t-butoxycarbonylamino-4-[2-chloro-4-(3-trifluoromethylphenoxy)phenyl]-2-methylbutan-1-ol

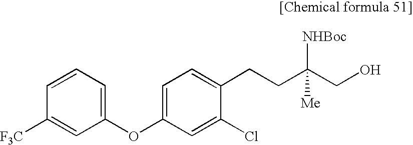

The compound of Example 3 was reacted in the same manner as in Example 11 to obtain an ester, which was then reacted in the same manner as in Example 15 to obtain the target product as a colorless oil.

¹H-NMR (CDCl₃, 400 MHz): δ 1.26 (3H, s), 1.45 (9H, s), 1.80-1.88 (1H, m), 2.05-2.12 (1H, m), 2.66-2.80 (2H, m), 3.68 (1H, d, J=11.6 Hz), 3.73 (1H, d, J=11.6 Hz), 4.70 (1H, br s), 6.86 (1H, dd, J=8.5, 2.5 Hz), 7.03 (1H, d, J=2.5 Hz), 7.13-7.16 (1H, m), 7.22-7.24 (2H, m), 7.37 (1H, d, J=7.9 Hz), 7.45 (1H, t, J=7.9 Hz).

FABMS (+): 474 [M+H]⁺.

### Example 18

### (R)-2-t-butoxycarbonylamino-4-[2-chloro-4-(3-trifluoromethylphenylthio)phenyl]-2-methylbutan-1-ol

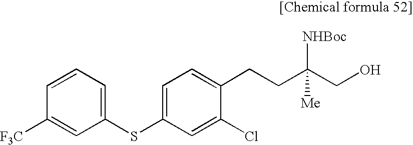

The compound of Example 4 was reacted in the same manner as in Example 11 to obtain an ester, which was then reacted in the same manner as in Example 15 to obtain the target product as a colorless oil.

¹H-NMR (CDCl₃, 400 MHz): δ 1.25 (3H, s), 1.44 (9H, s), 1.79-1.89 (1H, m), 2.05-2.13 (1H, m), 2.66-2.83 (2H, m), 3.68 (1H, d, J=12 Hz), 3.71 (1H, d, J=12 Hz), 4.69 (1H, br s), 7.20-7.23 (2H, m), 7.37-7.42 (3H, m), 7.45-7.50 (2H, m), 7.55 (1H, br s).

### Example 19

### (R)-2-t-butoxycarbonylamino-4-[2-chloro-4-(3-ethylphenylthio)phenyl]-2-methylbutan-1-ol

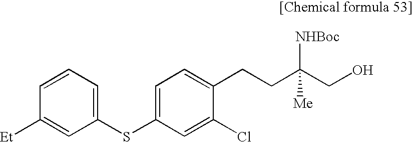

The compound of Example 5 was reacted in the same manner as in Example 11 to obtain an ester, which was then reacted in the same manner as in Example 15 to obtain the target product as a colorless oil.

¹H-NMR (CDCl₃, 400 MHz): δ 1.22 (3H, t, J=7.3 Hz), 1.24 (3H, s), 1.44 (9H, s), 1.77-1.85 (1H, m), 2.02-2.09 (1H, m), 2.62 (2H, q, J=7.3 Hz), 2.63-2.78 (2H, m), 3.64-3.73 (2H, m), 4.08 (1H, br), 4.68 (1H, br s), 7.10-7.17 (4H, m), 7.22-7.28 (3H, m).

ESIMS (+): 450 [M+H]⁺.

### Example 20

### (R)-2-t-butoxycarbonylamino-5-[2-chloro-4-(3-methylphenoxy)phenyl]-2-methylpentan-1-ol

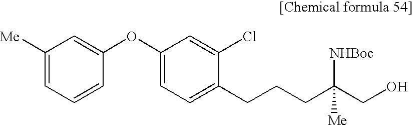

The compound of Example 6 was reacted in the same manner as in Example 11 to obtain an ester, which was then reacted in the same manner as in Example 15 to obtain the target product as a colorless oil.

¹H-NMR (CDCl₃, 400 MHz): δ 1.15 (3H, s), 1.43 (9H, s), 1.61-1.67 (3H, m), 1.83-1.87 (1H, m), 2.34 (3H, s), 2.70 (2H, t, J=7.0 Hz), 3.62-3.65 (2H, m), 4.57 (1H, s), 6.81-6.84 (3H, m), 6.94 (1H, d, J=7.3 Hz), 6.98 (1H, d, J=3.1 Hz), 7.15 (1H, d, J=7.9 Hz), 7.22 (1H, t, J=7.9 Hz).

ESIMS (+): 434 [M+H]⁺.

### Example 21

### (R)-2-t-butoxycarbonylamino-5-[2-chloro-4-(3-ethylphenylthio)phenyl]-2-methylpentan-1-ol

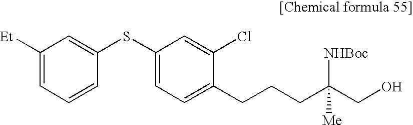

The compound of Example 7 was reacted in the same manner as in Example 11 to obtain an ester, which was then reacted in the same manner as in Example 15 to obtain the target product as a colorless oil.

¹H-NMR (CDCl₃, 400 MHz): δ 1.14 (3H, s), 1.22 (3H, t, J=7.3 Hz), 1.43 (9H, s), 1.54-1.70 (3H, m), 1.79-1.89 (1H, m), 2.62 (2H, q, J=7.3 Hz), 2.70 (2H, t, J=7.0 Hz), 3.57-3.66 (2H, m), 4.05 (1H, br), 4.55 (1H, br s), 7.10-7.17 (4H, m), 7.17-7.28 (3H, m).

ESIMS (+): 464 [M+H]⁺.

### Example 22

### (R)-2-t-butoxycarbonylamino-5-[2-chloro-4-(3-propylphenoxy)phenyl]-2-methylpentan-1-ol

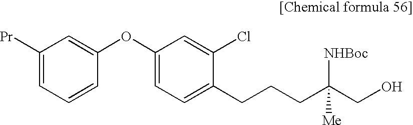

The compound of Reference Example 15 and (5S)-3,6-diethoxy-5-isopropyl-2-methyl-2,5-dihydropyrazine were reacted with in the same manner as in Example 1. The resultant compound was reacted in the same manner as in Example 11 to obtain an ester, which was then reacted in the same manner as in Example 15 to obtain the target product as a colorless oil.

¹H-NMR (CDCl₃, 400 MHz): δ 0.94 (3H, t, J=7.3 Hz), 1.15 (3H, s), 1.24-1.28 (2H, m), 1.43 (9H, s), 1.60-1.69 (3H, m), 1.80-1.90 (1H, m), 2.57 (2H, t, J=7.6 Hz), 2.70 (2H, t, J=7.6 Hz), 3.58-3.67 (2H, m), 4.11 (1H, br s), 4.58 (1H, br s), 6.79-6.85 (3H, m), 6.95 (1H, d, J=7.9 Hz), 6.99 (1H, d, J=2.8 Hz), 7.15 (1H, d, J=8.3 Hz), 7.24 (1H, t, J=7.9 Hz).

### Example 23

### (R)-2-t-butoxycarbonylamino-5-[2-chloro-4-(3-chlorophenylthio)phenyl]-2-methylpentan-1-ol

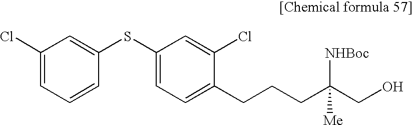

The compound of Example 8 was reacted in the same manner as in Example 11 to obtain an ester, which was then reacted in the same manner as in Example 15 to obtain the target product as a colorless oil.

¹H-NMR (CDCl₃, 400 MHz): δ 1.14 (3H, s), 1.43 (9H, s), 1.58-1.74 (3H, m), 1.79-1.92 (1H, m), 2.73 (2H, t, J=6.7 Hz), 3.61 (1H, d, J=12 Hz), 3.64 (1H, d, J=12 Hz), 4.08 (1H, br s), 4.57 (1H, br s), 7.17-7.27 (6H, m), 7.37 (1H, s).

ESIMS (+): 470 [M+H]⁺.

### Example 24

### (R)-2-t-butoxycarbonylamino-5-[2-fluoro-4-(3-trifluoromethylphenylthio)phenyl]-2-methylpentan-1-ol

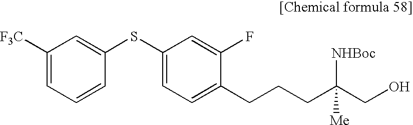

The compound of Example 12 was reacted in the same manner as in Example 15 to obtain the target product as a colorless oil.

¹H-NMR (CDCl₃, 400 MHz): δ 1.14 (3H, s), 1.42 (9H, s), 1.55-1.74 (3H, m), 1.75-1.85 (1H, m), 2.65 (2H, t, J=6.7 Hz), 3.58-3.64 (2H, m), 4.03 (1H, br s), 4.55 (1H, br s), 7.04 (1H, dd, J=9.8 Hz, 1.8 Hz), 7.10 (1H, dd, J=7.9 Hz, 1.8 Hz), 7.17 (1H, t, J=7.9 Hz), 7.38-7.50 (3H, m), 7.54 (1H, br s).

### Example 25

### (R)-2-t-butoxycarbonylamino-5-[2-chloro-4-(3-trifluoromethylphenylthio)phenyl]-2-propylpentan-1-ol

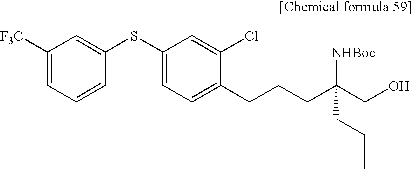

The compound of Example 14 was reacted in the same manner as in Example 15 to obtain the target product as a colorless oil.

¹H-NMR (CDCl₃, 400 MHz): δ 0.92 (3H, t, J=7.3 Hz), 1.42 (9H, s), 1.14-1.80 (8H, m), 2.72 (2H, t, J=7.3 Hz), 3.62 (1H, d, J=12 Hz), 3.66 (1H, d, J=12 Hz), 4.54 (1H, br s), 7.16-7.22 (2H, m), 7.39 (1H, s), 7.40-7.48 (3H, m), 7.55 (1H, br s).

FABMS (+): 532 [M+H]⁺.

### Example 26

### (R)-2-amino-5-[2-chloro-4-(3-trifluoromethylphenoxy)phenyl]-2-methylpentan-1-ol hydrochloride

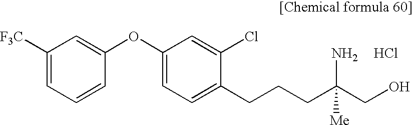

To the compound of Example 15 (6.50 g) was added a 10 w/w % hydrogen chloride solution in methanol (methanol containing hydrogen chloride, 67 mL), and the resultant mixture was stirred for 1 hour at room temperature, and then left overnight at room temperature. The solvent was then evaporated to obtain the target product (5.15 g) as a colorless amorphous.

¹H-NMR (DMSO-d₆, 400 MHz): δ 1.07 (3H, s), 1.46-1.64 (4H, m), 2.62-2.72 (2H, m), 3.31-3.36 (2H, m), 7.03 (1H, dd, J=7.9, 2.4 Hz), 7.20 (1H, d, J=2.4 Hz), 7.30 (1H, d, J=7.9 Hz), 7.34 (1H, s), 7.39 (1H, d, J=7.9 Hz), 7.52 (1H, d, J=7.9 Hz), 7.63 (1H, t, J=7.9 Hz).

HREIMS (+): 388.1281 (Calcd. for C₁₉H₂₁NClF₃O₂: 388.1291).

Optical Rotation: [α]_{D}²³-2.74 (c 0.63, CHCl₃).

### Example 27

### (R)-2-amino-5-[2-chloro-4-(3-trifluoromethylphenylthio)phenyl]-2-methylpentan-1-ol hydrochloride

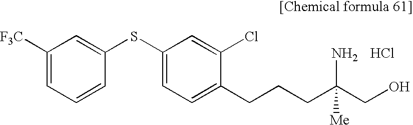

The compound of Example 16 was reacted in the same manner as in Example 26 to obtain the target product as a white powder.

¹H-NMR (DMSO-d₆, 400 MHz): δ 1.09 (3H, s), 1.49-1.63 (4H, m), 2.65-2.71 (2H, br s), 3.34 (1H, d, J=12 Hz), 3.38 (1H, d, J=12 Hz), 7.34 (1H, dd, J=7.9 Hz, 2.4 Hz), 7.41 (1H, d, J=7.9 Hz), 7.49 (1H, d, J=2.4 Hz), 7.55 (1H, d, J=7.9 Hz), 7.61 (1H, d, J=2.4 Hz), 7.67 (1H, d, J=7.9 Hz), 7.53-7.74 (3H, br s).

ESIMS (+): 404 [M+H]⁺.

Elemental Analysis Measured: C, 51.65%, H, 4.86%, N, 2.86%, Calcd. for C₁₉H₂ClF₃NOS.HCl: C, 51.82%, H, 5.04%, N, 3.18%.

Optical Rotation: [α]_{D}²³-3.45 (c 1.00, CHCl₃).

### Example 28

### (R)-2-amino-4-[2-chloro-4-(3-trifluoromethylphenoxy)phenyl]-2-methylbutan-1-ol hydrochloride

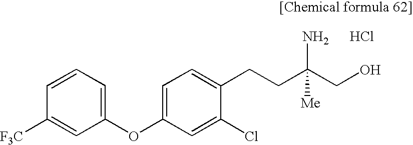

The compound of Example 17 was reacted in the same manner as in Example 26 to obtain the target product as a white powder.

¹H-NMR (DMSO-d₆, 400 MHz): δ 1.24 (3H, s), 1.70-1.80 (2H, m), 2.71 (2H, t, J=8.6 Hz), 3.44 (1H, dd, J=11 Hz, 4.9 Hz), 3.50 (1H, dd, J=11 Hz, 4.9 Hz), 5.54 (1H, t, J=4.9 Hz), 7.04 (1H, dd, J=8.6, 2.4 Hz), 7.21 (1H, d, J=2.4 Hz), 7.31 (1H, dd, J=8.6, 2.4 Hz), 7.35 (1H, br s), 7.41 (1H, d, J=8.6 Hz), 7.52 (1H, d, J=7.9 Hz), 7.63 (1H, t, J=7.9 Hz), 7.95 (3H, br s).

FABMS (+): 374 [M+H]⁺.

Elemental Analysis: Measured: C, 52.38%, H, 4.80%, N, 3.42%, Calcd. for C₁₈H₁₉ClF₃NO₂.HCl: C, 52.70%, H, 4.91%, N, 3.41%.

### Example 29

### (R)-2-amino-4-[2-chloro-4-(3-trifluoromethylphenylthio)phenyl]-2-methylbutan-1-ol hydrochloride

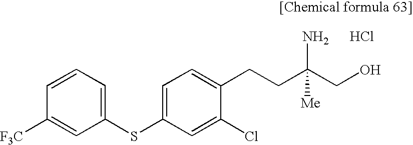

The compound of Example 18 was reacted in the same manner as in Example 26 to obtain the target product as a white powder.

¹H-NMR (DMSO-d₆, 400 MHz): δ 1.22 (3H, s), 1.66-1.83 (2H, m), 2.72 (2H, t, J=8.6 Hz), 3.42 (1H, dd, J=11.0, 7.9 Hz), 3.49 (1H, dd, J=11.0, 7.9 Hz), 5.54 (1H, t, J=4.9 Hz), 7.36 (1H, dd, J=7.9, 1.8 Hz), 7.42 (1H, d, J=7.9 Hz), 7.50 (1H, d, J=1.8 Hz), 7.53-7.64 (3H, m), 7.67 (1H, d, J=7.9 Hz), 7.82 (3H, br s).

FABMS (+): 390 [M+H]⁺.

Elemental Analysis Measured: C, 50.47%, H, 4.65%, N, 3.36%, Calcd. for C₁₈H₁₉ClF₃NOS.HCl: C, 50.71%, H, 4.73%, N, 3.29%.

Optical Rotation: [α]_{D}²⁷+5.78 (c 0.33, CHCl₃).

### Example 30

### (R)-2-amino-4-[2-chloro-4-(3-ethylphenylthio)phenyl]-2-methylbutan-1-ol hydrochloride

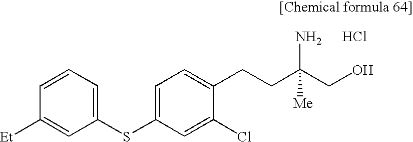

The compound of Example 19 was reacted in the same manner as in Example 26 to obtain the target product as a white powder.

¹H-NMR (DMSO-d₆, 400 MHz): δ 1.14 (3H, t, J=7.3 Hz), 1.22 (3H, s), 1.67-1.81 (2H, m), 2.59 (2H, q, J=7.3 Hz), 2.69 (2H, t, J=8.6 Hz), 3.42 (1H, dd, J=11.6, 5.5 Hz), 3.48 (1H, dd, J=11.6, 5.5 Hz), 5.52 (1H, t, J=4.9 Hz), 7.16-7.22 (2H, m), 7.26-7.27 (2H, m), 7.30-7.35 (2H, m), 7.93 (3H, br s).

ESIMS (+): 350 [M+H]⁺.

Elemental Analysis: Measured: C, 58.90%, H, 6.42%, N, 3.59%, Calcd. for C₁₉H₂₄ClNOS.HCl: C, 59.06%, H, 6.52%, N, 3.63%.

### Example 31

### (R)-2-amino-5-[2-chloro-4-(3-methylphenoxy)phenyl]-2-methylpentan-1-ol hydrochloride

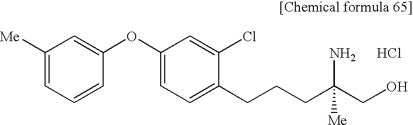

The compound of Example 20 was reacted in the same manner as in Example 26 to obtain the target product as a colorless amorphous.

¹H-NMR (DMSO-d₆, 400 MHz): δ 1.11 (3H, s), 1.57 (4H, brs), 2.29 (3H, s), 2.64 (2H, brs), 3.35-3.39 (2H, m), 5.45 (1H, t, J=4.9 Hz), 6.81 (1H, dd, J=8.6, 2.4 Hz), 6.85 (1H, s), 6.92 (1H, dd, J=8.6, 2.4 Hz), 6.99 (1H, d, J=8.6 Hz), 7.03 (1H, d, J=2.4 Hz), 7.28 (1H, t, J=8.6 Hz), 7.34 (1H, d, J=8.6 Hz), 7.77 (3H, brs).

HRESIMS (+): 334.15655 (Calcd. for C₁₉H₂₅ClNO₂: 334.15738).

Optical Rotation: [α]_{D}^{26.7}−5.75 (c 0.60, CHCl₃).

### Example 32

### (R)-2-amino-5-[2-chloro-4-(3-ethylphenylthio)phenyl]-2-methylpentan-1-ol hydrochloride

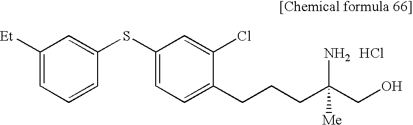

The compound of Example 21 was reacted in the same manner as in Example 26 to obtain the target product as a colorless oil.

¹H-NMR (DMSO-d₆, 400 MHz): δ 1.10 (3H, s), 1.15 (3H, t, J=7.3 Hz), 1.52-1.58 (4H, m), 2.59 (2H, q, J=7.3 Hz), 2.62-2.66 (2H, m), 3.32-3.39 (2H, m), 5.43 (1H, br), 7.15-7.22 (3H, m), 7.26 (2H, d, J=1.8 Hz), 7.32 (2H, dd, J=7.3, 1.8 Hz), 7.81 (3H, br s).

HRESIMS (+): 364.15051 (Calcd. for C₂₀H₂₇ClNOS: 364.15019).

### Example 33

### (R)-2-amino-5-[2-chloro-4-(3-propylphenoxy)phenyl]-2-methylpentan-1-ol hydrochloride

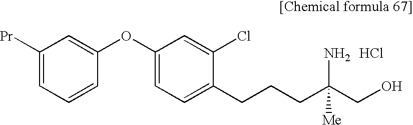

The compound of Example 22 was reacted in the same manner as in Example 26 to obtain the target product as a colorless amorphous.

¹H-NMR (DMSO-d₆, 400 MHz): δ 0.86 (3H, t, J=7.3 Hz), 1.11 (3H, s), 1.51-1.61 (6H, m), 2.53 (2H, t, J=7.3 Hz), 2.63 (2H, t, J=6.7 Hz), 3.34-3.42 (2H, m), 5.45 (1H, t, J=4.9 Hz), 6.81 (1H, ddd, J=7.9, 1.8, 0.9 Hz), 6.87 (1H, t, J=1.8 Hz), 6.91 (1H, dd, J=8.6, 2.4 Hz), 7.00 (1H, d, J=7.9 Hz), 7.02 (1H, d, J=2.4 Hz), 7.30 (1H, t, J=7.9 Hz), 7.34 (1H, d, J=8.6 Hz), 7.85 (3H, br s).

ESIMS (+): 362 [M+H]⁺.

HRESIMS (+): 362.19198 (Calcd. for C₂₁H₂₉ClNO₂: 362.18868).

Optical Rotation: [α]_{D}^{25.1}−4.46 (c 1.27, CHCl₃).

### Example 34

### (R)-2-amino-5-[2-chloro-4-(3-chlorophenylthio)phenyl]-2-methylpentan-1-ol hydrochloride

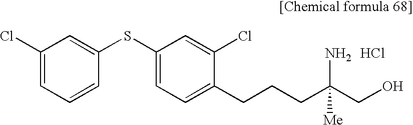

The compound of Example 23 was reacted in the same manner as in Example 26 to obtain the target product as a colorless amorphous.

¹H-NMR (DMSO-d₆, 400 MHz): δ 1.10 (3H, s), 1.49-1.64 (4H, m), 2.68 (2H, br s), 3.33 (1H, dd, J=12, 4.9 Hz), 3.38 (1H, dd, J=12, 4.9 Hz), 5.45 (1H, t, J=4.9 Hz), 7.26 (1H, dt, J=7.3, 1.8 Hz), 7.30-7.43 (5H, m), 7.45 (1H, d, J=1.8 Hz), 7.77 (3H, br s).

HREIMS (+): 370.0799 (Calcd. for C₁₈H₂₁Cl₂NOS: 370.0799).

Optical Rotation: [α]_{D}²⁷−3.81 (c 0.50, CHCl₃).

### Example 35

### (R)-2-amino-5-[2-fluoro-4-(3-trifluoromethylphenylthio)phenyl]-2-methylpentan-1-ol hydrochloride

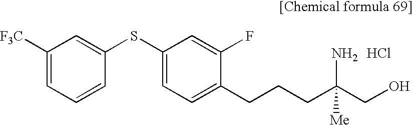

The compound of Example 24 was reacted in the same manner as in Example 26 to obtain the target product as a colorless amorphous.

¹H-NMR (DMSO-d₆, 400 MHz): δ 1.09 (3H, s), 1.48-1.61 (4H, m), 2.57-2.64 (2H, br s), 3.32 (1H, dd, J=11, 4.9 Hz), 3.37 (1H, dd, J=11, 4.9 Hz), 5.44 (1H, t, J=4.9 Hz), 7.20 (1H, dd, J=7.9, 1.8 Hz), 7.26 (1H, dd, J=9.8, 1.8 Hz), 7.37 (1H, t, J=7.9 Hz), 7.54-7.68 (4H, m), 7.74 (3H, br s).

HRESIMS (+): 388.1345 (Calcd. for C₁₉H₂₂F₄NOS: 388.1358).

Optical Rotation: [α]_{D}²⁴−3.23 (c 0.69, CHCl₃).

### Example 36

### (R)-2-amino-5-[2-chloro-4-(3-trifluoromethylphenylthio)phenyl]-2-propylpentan-1-ol hydrochloride

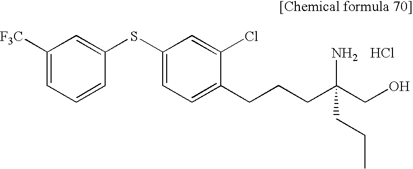

The compound of Example 25 was reacted in the same manner as in Example 26 to obtain the target product as a white powder.

¹H-NMR (DMSO-d₆, 400 MHz): δ 0.84 (3H, t, J=7.3 Hz), 1.20 (2H, q, J=7.3 Hz), 1.36-1.63 (6H, m), 2.68 (2H, t, J=7.3 Hz), 3.36 (2H, d, J=4.9 Hz), 5.40 (1H, d, J=4.9 Hz), 7.35 (1H, dd, J=7.9 Hz, 1.8 Hz), 7.42 (1H, d, J=7.9 Hz), 7.50 (1H, d, J=1.8 Hz), 7.55 (1H, d, J=7.9 Hz), 7.58-7.63 (2H, m), 7.67 (1H, d, J=7.9 Hz), 7.69 (3H, br s).

FABMS (+): 432 [M+H]⁺.

Elemental Analysis Measured: C, 53.46%, H, 5.62%, N, 2.98%, Calcd. for C₂₁H₂₅ClF₃NOS.HCl: C, 53.85%, H, 5.59%, N, 2.99%.

Optical Rotation: [α]_{D}²³+3.85 (c 0.63, CHCl₃).

### Example 37

### (R)-2-amino-5-[2-chloro-4-(3-trifluoromethylphenylthio)phenyl]-2-methylpentan-1-ol

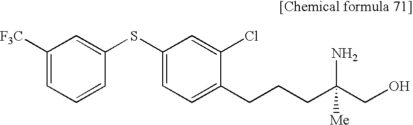

To a solution of the compound of Example 27 (9.3 g) in ethyl acetate (450 mL) was added saturated aqueous sodium hydrogen carbonate solution (450 mL), and the resultant solution was stirred at room temperature for 10 minutes. The organic layer was washed with water and saturated brine in that order, and then dried over anhydrous sodium sulfate. The solvent was evaporated, and the resultant residue was purified by NH-silica gel column chromatography (ethyl acetate:methanol=4:1) to obtain the target product (8.9 g) as a white powder.

¹H-NMR (DMSO-d₆, 400 MHz): δ 0.85 (3H, s), 1.21 (2H, br s), 1.28 (2H, t, J=8.6 Hz), 1.46-1.67 (2H, m), 2.65 (2H, t, J=8.6 Hz), 3.06 (2H, br s), 4.49 (1H, br s), 7.32 (1H, dd, J=7.9, 1.8 Hz), 7.40 (1H, d, J=9.8 Hz), 7.47 (1H, d, J=1.8 Hz), 7.54 (1H, dd, J=6.7, 1.8 Hz), 7.56-7.62 (2H, m), 7.65 (1H, dd, J=6.7, 1.8 Hz).

ESIMS (+): 404 [M+H]⁺.

Elemental Analysis Measured: C, 56.26%, H, 5.14%, N, 3.40%, Calcd. for C₁₉H₂₁ClF₃NOS: C, 56.50%, H, 5.24%, N, 3.47%.

### Example 38

### Diethyl 2-{3-[2-chloro-4-(3-trifluoromethylphenylthio)phenyl]propyl}-2-methylmalonate

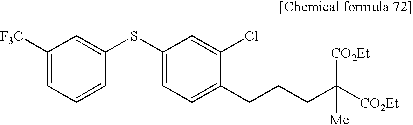

2-Chloro-1-(3-iodopropyl)-4-(3-trifluoromethylphenylthio)benzene and diethyl 2-methylmalonate were reacted according to the same procedures as in Example 152 of WO 04026817 to obtain the target product as a colorless oil.

¹H-NMR (CDCl₃, 400 MHz): δ 1.25 (6H, t, J=7.4 Hz), 1.40 (3H, s), 1.51-1.63 (2H, m), 1.90-1.97 (2H, m), 2.73 (2H, t, J=7.9 Hz), 4.17 (4H, q, J=7.4 Hz), 7.17-7.23 (2H, m), 7.38 (1H, d, J=2.2 Hz), 7.39-7.44 (2H, m), 7.45-7.50 (1H, m), 7.55 (1H, s).

EIMS (+): 502 [M]⁺.

### Example 39

### (±)-5-[2-chloro-4-(3-trifluoromethylphenylthio)phenyl]-2-ethoxycarbonyl-2-methylpentanoic acid

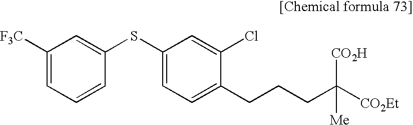

To a solution of the compound of Example 38 (16.8 g) in ethanol (167 mL) was added potassium hydroxide (2.40 g), and the resultant solution was stirred at 50° C. for 24 hours. To the reaction solution was added water, neutralized with 2 mol/L aqueous hydrochloric acid, and extracted with ethyl acetate. The organic layer was washed with water and saturated brine in that order, and then dried over anhydrous sodium sulfate. The solvent was evaporated, and the resultant residue was purified by silica gel column chromatography (hexane:ethyl acetate=1:1) to obtain the target product (11.2 g) as a colorless oil.

¹H-NMR (CDCl₃, 400 MHz): δ 1.26 (3H, t, J=7.4 Hz), 1.47 (3H, s), 1.55-1.66 (2H, m), 1.87-2.06 (2H, m), 2.73 (2H, t, J=7.9 Hz), 4.22 (2H, q, J=7.4 Hz), 7.18 (1H, d, J=7.9 Hz), 7.20 (1H, dd, J=7.9, 1.8 Hz), 7.38 (1H, d, J=1.8 Hz), 7.39-7.44 (2H, m), 7.45-7.50 (1H, m), 7.54 (1H, s).

ESIMS (+): 475 [M+H]⁺.

### Example 40

### Ethyl(±)-5-[2-chloro-4-(3-trifluoromethylphenylthio)phenyl]-2-methoxycarbonylamino-2-methylpentanoate

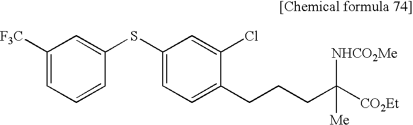

To a solution of the compound of Example 39 (15.8 g) in benzene (166 mL) was added diphenylphosphoryl azide (7.86 mL) and triethylamine (6.01 mL), and the resultant solution was heated to reflux for 1.5 hours. The temperature of the reaction solution was returned to room temperature, and methanol (20 mL) was added dropwise over 20 minutes. The resultant solution was heated to reflux for 30 minutes, and then further sodium methoxide (3.58 g) was added. The resultant solution was heated to reflux for 1.5 hours. To the reaction solution was added saturated aqueous ammonium chloride, and extracted with ethyl acetate. The organic layer was washed with water and saturated brine in that order, and then dried over anhydrous sodium sulfate. The solvent was evaporated, and the resultant residue was purified by silica gel column chromatography (hexane:ethyl acetate=5:1) to obtain the target product (15.6 g) as a colorless oil.

¹H-NMR (CDCl₃, 400 MHz): δ 1.25 (3H, t, J=7.3 Hz), 1.32-1.47 (1H, m), 1.52-1.67 (1H, m), 1.57 (3H, s), 1.80-1.90 (1H, m), 2.20-2.37 (1H, m), 2.62-2.76 (2H, m), 3.64 (3H, s), 4.15-4.25 (2H, m), 5.62 (1H, br s), 7.16 (1H, d, J=7.9 Hz), 7.20 (1H, dd, J=7.9, 1.8 Hz), 7.38 (1H, d, J=1.8 Hz), 7.40-7.44 (2H, m), 7.45-7.50 (1H, m), 7.55 (1H, s).

ESIMS (+): 504 [M+H]⁺.

### Example 41

### (±)-5-[2-chloro-4-(3-trifluoromethylphenylthio)phenyl]-2-methoxycarbonylamino-2-methylpentan-1-ol

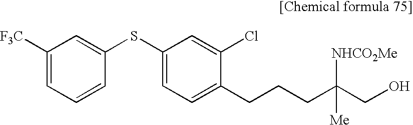

To a solution of the compound of Example 40 (15.6 g) in THF (249 mL) was added under ice cooling lithium borohydride (3.75 g), and then ethanol (16.6 mL) was added dropwise. The resultant solution was then stirred for 1 hour under ice cooling. To the reaction solution was added 10% aqueous citric acid, extracted with ethyl acetate, washed with water and saturated brine in that order, and then dried over anhydrous sodium sulfate. The solvent was evaporated, and the resultant residue was purified by silica gel column chromatography (hexane:ethyl acetate=1:1) to obtain the target product (12.9 g) as a colorless oil.

¹H-NMR (CDCl₃, 400 MHz): δ 1.18 (3H, s), 1.54-1.74 (3H, m), 1.78-1.89 (1H, m), 2.73 (2H, t, J=7.9 Hz), 3.63 (3H, s), 3.56-3.70 (2H, m), 4.23 (1H, br s), 7.17-7.22 (2H, m), 7.38-7.50 (4H, m), 7.54 (1H, s).

ESIMS (+): 462 [M+H]⁺.

### Example 42

### (±)-2-t-butoxycarbonylamino-5-[2-chloro-4-(3-trifluoromethylphenylthio)phenyl]-2-methylpentan-1-ol

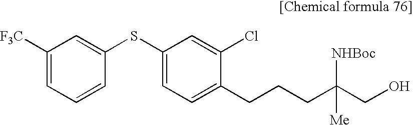

To a solution of the compound of Example 41 (12.9 g) in THF (60 mL) and methanol (120 mL) was added under ice cooling 5 mol/L aqueous potassium hydroxide solution (60 mL), and the resultant solution was heated to reflux for 86 hours. To the reaction solution was added water, extracted with ethyl acetate, washed with water and saturated brine in that order, and then dried over anhydrous sodium sulfate. The extract was concentrated, the residue was dissolved in 1,4-dioxane (279 mL), and the resultant solution was charged with di-tert-butoxydicarbonate (9.13 g). The solution was stirred at room temperature for 2 hours and then left to stand at room temperature overnight. The reaction solution was added water, extracted with ethyl acetate, washed with water and saturated brine in that order, and then dried over anhydrous sodium sulfate. The solvent was evaporated, and the resultant residue was purified by silica gel column chromatography (hexane:ethyl acetate=2:1) to obtain the target product (13.0 g) as a colorless oil.

¹H-NMR (CDCl₃, 400 MHz): δ 1.14 (3H, s), 1.42 (9H, s), 1.53-1.74 (3H, m), 1.79-1.92 (1H, m), 2.74 (2H, t, J=7.9 Hz), 3.58-3.69 (2H, m), 4.05 (1H, br s), 4.57 (1H, br s), 7.20-7.22 (2H, m), 7.38-7.50 (4H, m), 7.54 (1H, s).

ESIMS (+): 504 [M+H]⁺.

### Examples 43 and 44

### (+)-2-t-butoxycarbonylamino-5-[2-chloro-4-(3-trifluoromethylphenylthio)phenyl]-2-methylpentan-1-ol and (−)-2-t-butoxycarbonylamino-5-[2-chloro-4-(3-trifluoromethylphenylthio)phenyl]-2-methylpentan-1-ol

The compound of Example 42 was subjected to optical resolution using high performance liquid chromatography (CHIRALCEL OJ-H, hexane:isopropanol: diethylamine=98:2:0.1 (v/v), measurement wavelength:UV 278 nm, flow rate:1.0 mL/min). From the pre-elution portion, an [α]_{D}²⁵+15.08 (c 0.63, CHCl₃) colorless oil was obtained (Example 43), and from the post-elution portion, an [α]_{D}²⁶−13.91 (c 0.63, CHCl₃) colorless oil was obtained (Example 44).

### Example 45

### (−)-2-t-butoxycarbonylamino-5-[2-chloro-4-(3-trifluoromethylphenylthio)phenyl]-2-methylpentan-1-ol hydrochloride

The compound of Example 43 was reacted in the same manner as in Example 26 to obtain the target product as a white powder.

ESIMS (+): 404 [M+H]⁺.

Optical Rotation: [α]_{D}²⁵−4.48 (c 1.00, CHCl₃).

### Test Example

### Effect on a Mouse Experimental Autoimmune Encephalomyelitis (EAE) Model

The test was carried out in accordance with the method described in Current Protocols in Immunology, Chapter 15. That is, Myelin Basic Protein, Mouse (MBP, Sigma M 2941) was dissolved in physiological saline to a concentration of 1 mg/mL and used as an antigen liquid. M. tuberculosis Des. H37RA was added to Adjuvant Incomplete Freund (IFA, Difco 263910) to a concentration of 4 mg/mL, thoroughly mixed by applying an ultrasonic wave and used as Complete Freund Adjuvant (CFA). The antigen liquid and CFA were mixed to a ratio of 1:1, and an ultrasonic treatment was applied thereto, thereby preparing an immunization emulsion.

Under un-anesthesia, legs and arms of each mouse were fixed using a string, and dorsal hair was shaven to expose the skin. A 0.1 mL per animal of the immunization emulsion was intradermally injected by dividing into three positions (one central position of the back along the median line and two lumbar positions sandwiching the median line). Thereafter, 0.1 mL of a Pertussis Toxin (Sigma) solution was intraperitoneally administered (400 ng/body). Two days later, 0.1 mL of the Pertussis Toxin solution was again intraperitoneally administered. After commencement of the test, clinical symptoms of the mice were observed once a day for 6 weeks, and scoring was carried out in accordance with the following criteria. In this connection, the symptom evaluators and drug administrators were separated. The scoring process was performed under blinded manner. In addition, when an individual died, the score on and after that day was regarded as 5.

TABLE 1

| Score | Clinical signs |
|---|---|
| 0 | Normal mouse; no overt signs of disease |
| 1 | Limp tail or hind limb weakness but not both |
| 2 | Limp tail and hind limb weakness |
| 3 | Partial hind limb paralysis |
| 4 | Complete hind limb paralysis |
| 5 | Moribund state; death |

The compound of Example 27 (compound 27) was administered by dissolving in ultrapure water. The compound 27 was dissolved in ultrapure water to a concentration of 0.1 mg/mL and stored in a refrigerator (set-up temperature 4° C., shading). This was stored for 10 days at the maximum. Regarding the 0.01 to 0.3 mg/kg administration liquids, each drug liquid of the intended concentration (0.03, 0.01, 0.003 or 0.001 mg/mL) was prepared in each day of carrying out the administration by diluting 0.1 mg/mL of the drug liquid with ultrapure water. The drug liquid was orally administered at a ratio of 0.1 mL per 10 g body weight. Ultrapure water was administered to the control group in the same manner. The administration was carried out once a day for every day of from the 5th day to 41st day after the immunization. In this connection, the test was carried out by N=9 for the 0.3 mg/kg administration group and by N=10 for the rest of the administration groups.

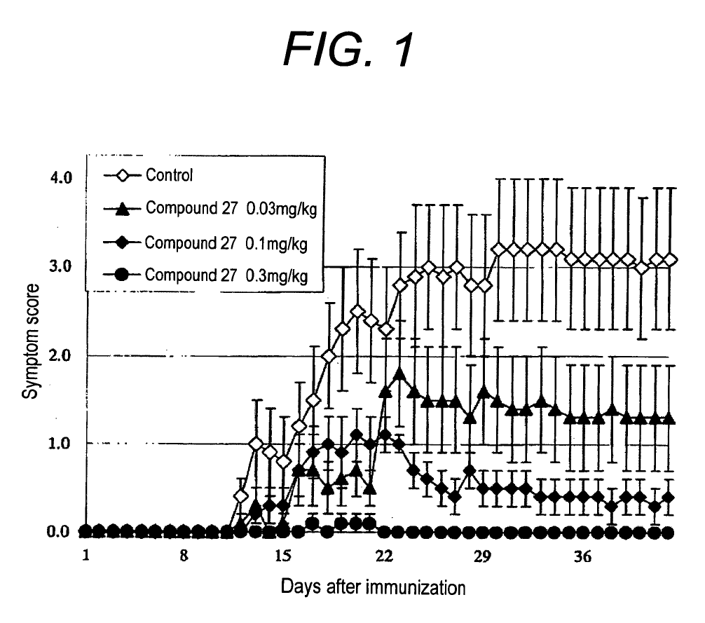

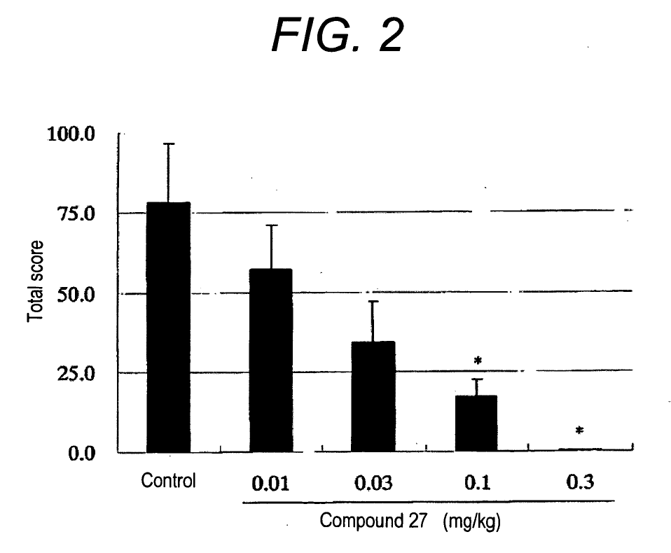

A change in the morbid state symptom score after immunization is shown in FIG. 1, and the total of symptom scores in each group is shown in FIG. 2. Oral administration of the compound 27 inhibited the onset of disease dose-dependently, and showed maximum reaction by the 0.3 mg/kg administration.

### Formulation Example

### Composition

| | |
|---|---|
| Compound 27 | 0.1 mg |
| D-mannitol | 247.5 mg |
| Magnesium stearate | 2.5 mg |

A mixed powder was produced by mixing the compound 27 with D-mannitol and further mixing magnesium stearate therewith. A capsule preparation was produced by filling this mixed powder in a capsule.

### INDUSTRIAL APPLICABILITY

The compound of the invention enables to provide a pharmaceutical preparation which is useful for the treatment or prevention of human demyelinating diseases, particularly multiple sclerosis.

1-7. (canceled)

8. A method of treating or preventing a demyelinating disease, the method comprising administrating an effective amount of an amino alcohol derivative represented by the general formula (1), or a pharmaceutically acceptable salt or hydrate thereof: 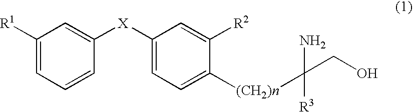
wherein R¹ represents a chlorine atom or a straight-chain alkyl group having 1 to 3 carbon atoms or a trifluoromethyl group, R² represents a fluorine atom or a chlorine atom, R³ represents a straight-chain alkyl group having 1 to 3 carbon atoms, X represents an oxygen atom or a sulfur atom, and n denotes 2 or 3.

9. The method according to claim 8, wherein the compound represented by the general formula (1) is a compound represented by the general formula (1a), 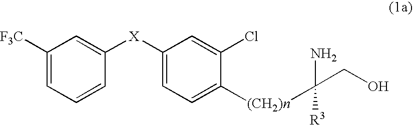
wherein R³ represents a straight-chain alkyl group having 1 to 3 carbon atoms, X represents an oxygen atom or a sulfur atom, and n denotes 2 or 3.

10. The method according to claim 8, wherein R³ in the general formulae (1) is a methyl group.

11. The method according to claim 9, wherein R³ in the general formulae (1a) is a methyl group.

12. The method according to claim 8, wherein the compound represented by the general formula (1) is,
1) (R)-2-amino-5-[2-chloro-4-(3-trifluoromethylphenoxy)phenyl]-2-methylpentan-1-ol,
2) (R)-2-amino-5-[2-chloro-4-(3-trifluoromethylphenylthio)phenyl]-2-methylpentan-1-ol,
3) (R)-2-amino-4-[2-chloro-4-(3-trifluoromethylphenoxy)phenyl]-2-methylbutan-1-ol,
4) (R)-2-amino-4-[2-chloro-4-(3-trifluoromethylphenylthio)phenyl]-2-methylbutan-1-ol,
5) (R)-2-amino-5-[2-chloro-4-(3-ethylphenylthio)phenyl]-2-methylpentan-1-ol,
6) (R)-2-amino-5-[2-fluoro-4-(3-trifluoromethylphenylthio)phenyl]-2-methylpentan-1-ol, or
7) (R)-2-amino-5-[2-chloro-4-(3-trifluoromethylphenylthio)phenyl]-2-propylpentan-1-ol.

13. The method according to claim 8, wherein the compound represented by the general formula (1) in an optically active amino alcohol derivative, or a pharmaceutically acceptable salt or hydrate thereof, which is obtainable by:
allowing a compound represented by the general formula (2) and a compound represented by the general formula (10) to react each other in the presence of a base, 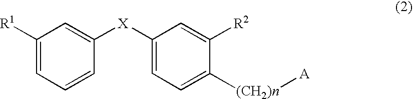
wherein R¹ represents a chlorine atom or a straight-chain alkyl group having 1 to 3 carbon atoms or a trifluoromethyl group, R² represents a fluorine atom or a chlorine atom, A represents a halogen atom, X represents an oxygen atom or a sulfur atom, and n denotes 2 or 3, 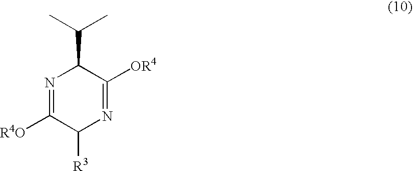
wherein R³ represents a straight-chain alkyl group having 1 to 3 carbon atoms and R⁴ represents an alkyl group having 1 to 6 carbon atoms,
subjecting the resultant product to acidolysis,
protecting a nitrogen atom with a t-butoxycarbonyl group,
reducing the resultant protected compound, and
deprotecting the nitrogen atom of the resultant reduced compound.

14. The method according to claim 8, wherein the compound represented by the general formula (1) is,
1) (−)-2-amino-5-[2-chloro-4-(3-trifluoromethylphenoxy)phenyl]-2-methylpentan-1-ol,
2) (−)-2-amino-5-[2-chloro-4-(3-trifluoromethylphenylthio)phenyl]-2-methylpentan-1-ol,
3) (+)-2-amino-4-[2-chloro-4-(3-trifluoromethylphenylthio)phenyl]-2-methylbutan-1-ol,
4) (−)-2-amino-5-[2-fluoro-4-(3-trifluoromethylphenylthio)phenyl]-2-methylpentan-1-ol, or
5) (+)-2-amino-5-[2-chloro-4-(3-trifluoromethylphenylthio)phenyl]-2-propylpentan-1-ol.
